(19) United States
(12) Patent Application Publication
(10) Pub. No.: US 2017/0145061 A1
(43) Pub. Date: May 25, 2017

(54) ENGINEERED BACTEROIDES OUTER MEMBRANE VESICLES

(71) Applicant: Massachusetts Institute of Technology, Cambridge, MA (US)

(72) Inventors: TIMOTHY KUAN-TA LU, Cambridge, MA (US); Mark K. Mimee, Cambridge, MA (US); Juliane Ripka, Freiburg (DE)

(73) Assignee: Massachusetts Institute of Technology, Cambridge, MA (US)

(21) Appl. No.: 15/355,809

(22) Filed: Nov. 18, 2016

Related U.S. Application Data
(60) Provisional application No. 62/413,398, filed on Oct. 26, 2016.
(60) Provisional application No. 62/257,849, filed on Nov. 20, 2015.

(51) Int. Cl. C07K 14/195; C12N 15/74; A61K 38/20; C07K 14/54; A61K 38/16; C12N 15/62; A61K 35/74

(52) U.S. Cl. CPC C07K 14/195; C12N 15/62; C12N 15/74; A61K 35/74; C07K 14/5428; A61K 38/164; A61K 38/2066; C07K 2319/50; A61K 2035/11

(57) ABSTRACT

The present disclosure provides, in some aspects, versatile intestinal protein delivery systems deploying engineered human gut commensals of the Bacteroides species to secrete heterologous, therapeutic proteins via outer membrane vesicles (OMVs).

### RELATED APPLICATIONS

This application claims the benefit under 35 U.S.C. §119(e) of U.S. provisional application No. 62/257,849, filed Nov. 20, 2015, and U.S. provisional application No. 62/413,398, filed Oct. 26, 2016, each of which is incorporated by reference herein in its entirety.

### FEDERALLY-SPONSORED RESEARCH

This invention was made with government support under Grant No. OD008435 awarded by National Institutes of Health. The government has certain rights in the invention.

### BACKGROUND

The targeted intestinal delivery of therapeutic proteins by genetically engineered live bacteria is an emerging field of research with broad potential in the treatment of human diseases. The conventional delivery of protein drugs like anti-inflammatory cytokines to the intestine is challenging as they are unstable when administered orally, or require high doses with severe side effects if administered systemically.

### SUMMARY

Engineered microbes overcome drawbacks of conventional protein delivery strategies by releasing protein drugs in close proximity of their site of action. Provided herein is a versatile intestinal protein delivery system deploying engineered human gut commensals of the Bacteroides species to secrete heterologous, therapeutic proteins via outer membrane vesicles (OMVs). Delivery via OMVs prevents cargo from dilution and proteolytic degradation. The stable and abundant intestinal colonization by bacteria of the beneficial genus Bacteroides particularly qualifies them as long-term therapeutics.

As shown in the working examples provided herein, heterologous proteins were secreted via Bacteroides OMVs by genetic fusion to small peptide tags derived from proteins naturally occurring in B. thetaiotaomicron and B. ovatus OMVs. First, three OMV proteins (BT1491, BT3238, BACOVA_04502) that target the reporter protein NanoLuc to B. thetaiotaomicron OMVs when fused to its N-terminus were identified. A bioinformatics analysis predicted that the proteins were lipoproteins containing N-terminal signal peptides with signal peptidase II cleavage sites. Second, truncations of the OMV proteins were generated to determine the minimal and optimal length for secretion. Peptide tags of 25-35 amino acids efficiently translocated functional NanoLuc to OMVs in B. thetaiotaomicron, B. fragilis, and B. vulgatus. Further, the anti-inflammatory molecule IL-10 was successfully secreted into the supernatants of B. thetaiotaomicron and B. vulgatus cultures at concentrations of up to 50 ng/mL. IL-10 was identified in purified OMVs at concentrations of 0.3 ng/mL. Additionally, stable intestinal colonization of mice was shown in an experimental model of inflammatory bowel disease with B. thetaiotaomicron, B. fragilis, and B. vulgatus, which had no detrimental impact on gut inflammation.

IL-10 has previously been shown to ameliorate inflammation in experimental animal models of inflammatory bowel disease. Thus, the production of this mammalian immunomodulatory protein and its secretion via outer membrane vesicles of the Bacteroides spp. is a candidate for the development of novel long-term therapies for inflammatory gut diseases. The treatment generally requires low doses and primarily avoids systemic side effects due to its targeted local delivery. Further, the treatment is applicable to a wide range of therapeutic protein drugs for treatment of various intestinal disorders.

These and other embodiments of the present disclosure will be described in greater detail herein.

### BRIEF DESCRIPTION OF DRAWINGS

FIG. 1 shows the structure of the Gram-negative cell envelope and outer membrane vesicles (OMVs). The bottom schematic illustrates the cell envelope of Gram-negative bacteria consisting of 3 compartments: the inner (cytoplasmic) membrane (IM) composed of phospholipids, the asymmetrical outer membrane (OM) comprising an interior leaflet of phospholipids and an exterior leaflet of lipopolysaccharide (LPS), as well as the periplasmic space in between, containing a peptidoglycan (PG) layer and periplasmic proteins. Envelope stability is mediated by various crosslinks including the Braun's lipoprotein (Lpp) linking the OM with the PG layer and the Tol-Pal (peptidoglycan-associated lipoprotein) complex spanning the cell envelope from the OM across the PG layer to the IM. The top right schematic depicts OMV budding from the OM, enclosing soluble periplasmic contents, including PG, enzymes, and nucleic acids as well as outer membrane proteins. The bottom schematic also illustrates major factors promoting OMV biogenesis: reduced Lpp-PG crosslinks loosen the OM; accumulation of envelope components or misfolded proteins turgor pressure (indicated by arrows); charge repulsion between OM constituents (indicated by two-headed arrows) cause membrane curvature, e.g., by particular types of LPS like the highly charged B-band LPS as opposed to the less charged A-band LPS in P. aeruginosa. The top left image shows scanning electron micrographs of OMVs budding from the surface of B. thetaiotaomicron. Arrows indicate bacterial membrane protrusions (left), marking the initiation and subsequent vesicle formation (right). Scale bars 100 nm. Micrograph from (179).

FIG. 2 shows mechanisms of OMV cargo delivery to host cells. (Panel A) Cargo anchored to the outer membrane can directly interact with receptors on host cells and cause an intracellular effect. (Panel B) Cargo loaded in the OMV lumen can be released by OMV lysis and subsequently diffuse to its target site. (Panel C) Membranes of OMV and host cell can fuse leading to cargo release in the cell cytoplasm. (Panel D) OMVs can be internalized as a whole entity by clathrin-mediated or caveolin-mediated endocytosis (non-phagocytic cells) or phagocytosis (phagocytic cells). Membrane fusion and endocytosis often depend on OMV-receptor binding before internalization.

FIG. 3 shows the fusion of NL to OMV proteins enriches NL in OMV-containing culture supernatants. Fusion proteins of NL with BT1491, BT3238, and BACOVA_04502, respectively, were expressed in B. thetaiotaomicron (TYG medium) and cells were separated from OMV-containing supernatants by centrifugation. Luciferase activities of supernatant and cell lysates are reported as RLU normalized by OD600 at the time of harvest (RLU/OD600). Depicted are means+SD of three independent biological replicates. Fold activities of tagged compared to untagged NL in supernatants are indicated above the bars. NL, NanoLuc; RLU, Relative Luminescence Units.

FIG. 4 shows BT1491, BT3238, and BACOVA_04502 are predicted OM lipoproteins with N-terminal signal peptides SPII cleavage site. Top, Multiple sequence alignment of the first 70 N-terminal amino acids of BT_1491, BT_3238, and BACOVA_04502 prelipoprotein precursors by T-Coffee software (www.ebi.ac.uk/Tools/msa/tcoffee/). Similarities between the sequences are highlighted, where (*), (:), and (.) denote decreasing levels of similarity. Acidic amino acids (D, E); basic amino acids (K, R, H); hydrophobic amino acids (M, A, I, V, L, W, F, P); polar amino acids (G, S, C, T, Q, N, Y). The sequences, from top to bottom, correspond to SEQ ID NO: 1-3. Bottom, The schematic representation below the alignment depicts the SPII cleavage site before a conserved cysteine residue, which separates the mature lipoprotein from the signal peptide as predicted by LipoP software. SPII, signal peptidase II.

FIGS. 5A-5C show a majority of OMV-associated proteins are SPII lipoproteins. In FIG. 5A, 61 proteins found in B. thetaiotaomicron (Bt) OMVs by Elhenawy et al. (74) and BACOVA_04502 were examined for signal peptidase I and II cleavage sides using LipoP software (cbs.dtu.dk/services/LipoP/). Percentages of proteins with signal peptidase I cleavage site (SPI), SPII, or no signal peptide are shown, respectively. The absolute numbers of proteins are shown in brackets. FIGS. 5B-5C show sequence logos of N-terminal lipoprotein sequences indicating 4 aa before and 6 aa after the cleavage site were generated for OMV lipoproteins (31 proteins from A) and ‘other lipoproteins’ using the WebLogo 3 software (weblogo.threeplusone.com/create.cgi). Shown are probabilities, where the height of symbols indicates the relative frequency of each amino acid at that position. FIG. 5C shows ‘other lipoproteins’ including 3 lipoproteins exclusively found in the outer membrane and 26 lipoproteins derived from UniProt database for which LipoP predicted SPII-cleavable signal peptides and which were not detected in OMVs. Acidic amino acids (D, E); basic amino acids (K, R, H); hydrophobic amino acids (M, A, I, V, L, W, F, P); polar amino acids (G, S, C, T, Q, N, Y).

FIGS. 6A-6D show truncated tags derived from the N-termini of OMV proteins are sufficient for efficient enrichment of NL in supernatants. Fusion proteins of NL with C-terminally truncated BT_1491 (FIG. 6A), BT_3238 (FIG. 6B), and BACOVA_04502 (FIG. 6C), respectively, were expressed in B. thetaiotaomicron (TYG medium). Cells were separated from OMV-containing supernatant by centrifugation. Luciferase activities of supernatant and cell lysates are reported as relative light units normalized by OD600 of liquid cultures (RLU/OD600). Depicted are means+SD of at least three independent biological replicates. Fold activities of the truncation used in the following experiments compared to untagged or FL tags in supernatants are indicated above the bars. Δx means x N-terminal amino acids. A schematic depiction of the fusion proteins can be found below each graph. Signal peptides predicted by LipoP are depicted in dark gray and the parts remaining in the mature proteins in light gray. Predicted signal peptidase II cleavage sites and residue numbers are indicated by arrows. FL, full length protein; NL, NanoLuc; RLU, Relative Luminescence Units. The sequences in FIG. 6A, from top to bottom, correspond to SEQ ID NO: 4-9. The sequences in FIG. 6B, from top to bottom, correspond to SEQ ID NO: 10-12. The sequences in FIG. 6C, from top to bottom, correspond to SEQ ID NO: 13-16. The His6 tag, H₆, corresponds to SEQ ID NO: 49. FIG. 6D shows that altering amino acid composition in OMV proteins can modulate secretion. The amino acids in the ‘+2’ or ‘+3’ position of BACOVA_04502 were exhaustively changed to each amino acid. Supernatants of the mutant variants as well as wild-type BACOVA_04502 were collected and assayed for luminescence activity. Data is depicted as the fold change in supernatant luminescence as compared with wild-type BACOVA_04502 (wild-type sequence is ‘SN’).

FIGS. 7A-7C show OMV proteins/tags enrich NL in purified B. thetaiotaomicron OMVs. FIG. 7A shows an electron microscopy micrograph of OMVs released by B. thetaiotaomicron. Vesicles were purified by high-speed centrifugation (70,000×g) of filtered (0.45 μm), cell-free culture supernatant. FIG. 7B shows OMV protein/tag-NanoLuc fusion proteins expressed in B. thetaiotaomicron and purified OMVs assayed for luciferase activity. Luciferase activity is reported as relative light units normalized by total protein content (RLU/μg). Shown are means+SD of at least three independent biological replicates. Significance by unpaired t test analysis and fold NL activities compared to NL are shown. * P≦0.05; ** P≦0.01; *** P≦0.001 **** P≦0.0001. FIG. 7C shows Western blot analysis of fusion proteins in purified OMVs detected with anti-His antibody. 4 μg of purified OMV proteins (as determined by total protein concentration using bradford assay) were loaded in each lane. Molecular weight (MW) ladder is indicated on the left. Arrow points at BACOVA_04502-NL band. Blot is representative for three independent experiments.

FIG. 8 shows that OMV tags induce NL translocation into OMVs of B. fragilis and B. vulgatus. Fusion proteins of OMV tags and NL were expressed in human feces isolates B. thetaiotaomicron (Bt) and B. fragilis (Bf), or murine isolate B. vulgatus (Bvm). Luciferase activity of purified OMVs was determined and is reported as relative light units normalized by total protein content (RLU/μg). Depicted are means+SD of three independent biological replicates (Bt, Bf) or single measurements (Bvm).

FIGS. 9A-9E show proteinase K protection assay of OMV-tag-NL fusion proteins in purified OMVs of B. thetaiotaomicron. FIGS. 9A-9C show OMVs of B. thetaiotaomicron expressing fusion proteins of NL with OMV tags treated with 0.1 mg mL⁻¹ proteinase K (PK) and/or 1% Triton X-100 (TritonX) as indicated to disrupt vesicle integrity for 45 min, 3 h, 9 h, and 24 h at 37° C. The relative luminescence shown is the ratio of the activity after proteinase K and/or TritonX treatment compared with the activity of untreated samples. Mean and individual samples of 1-3 independent experiments are depicted. FIG. 9D is a Western blot analysis showing proteinase K accessibility of fusion proteins in OMVs treated with 0.1 mg mL⁻¹ proteinase K (PK) and 1% SDS as indicated for 30 min at 37° C. 5 μg OMVs (as determined by total protein concentration using the Bradford protein assay) were loaded in each lane. Fusion proteins were detected with anti-His antibody. Molecular weight (MW) ladder is marked at left. Blot is representative for three independent experiments. FIG. 9E shows a Western blot analysis of BT1491Δ25 fusion protein after treatment of OMVs with indicated PK concentrations for the indicated time. NL, NanoLuc; PK, Proteinase K; MW, Molecular Weight.

FIGS. 10A-10D show IL-10 concentrations in concentrated culture supernatants and OMVs of B. thetaiotaomicron (Bt) and B. vulgatus mouse isolate (Bvm). ELISA analysis of murine IL-10 (mIL-10) concentrations [mIL-10] in concentrated culture supernatants fractioned by weight (>10 and >100 kDa) (FIGS. 10A-10B) and purified OMVs (FIGS. 10C-10D) of B. thetaiotaomicron (FIGS. 10A, 10C) and B. vulgatus (FIGS. 10B, 10D) expressing fusion proteins of mIL-10 with OMV tags. OMV tag-NL fusions and untagged mIL-10 were used as negative controls. mIL-10 concentrations are depicted as pg per μg of total protein content as determined by bradford assay. In FIGS. 10A-10B, supernatants were concentrated using filter tubes with either 10 kDa or 100 kDa cut-off membranes. Shown are means+SD of 3 or 4 independent experiments. Fold increases of [mIL-10] are indicated for OMV-tagged compared to untagged mIL-10 in >100 kDa concentrates. FIGS. 10C-10D show the means of [mIL-10] in by high-speed centrifugation purified OMVs from two independent experiments (Bt) and one experiment (Bvm), respectively.

FIG. 11 presents an experimental outline for the in vivo colonization of engineered Bacteroides species in a DSS-induced colitis mouse model. Specific-pathogen-free (SPF) C57BL/6 mice were treated with metronidazole and ciprofloxacin for 7 days. 2 days after cessation of antibiotic treatment, engineered B. thetaiotaomicron, B. fragilis, or B. vulgatus mouse isolate expressing the BT1491Δ25-NL fusion protein (Bt-BT1491Δ25-NL, Bf-BT1491Δ25-NL, Bvm-BT1491Δ25-NL) were gavaged orally. 6 days after Bt/Bfgavage and 3 days after Bvm gavage, respectively, mice were treated with 3% dextran sulfate sodium (DSS) and IPTG in drinking water for 7 days. At the end of the study, mice were sacrificed and feces and colon samples were collected for further analysis.

FIGS. 12A-12B show the colonization of mice with Bacteroides spp., producing BT1491Δ25-NL fusion proteins. Mice were orally gavaged on day 0 with 5×10⁸ CFU of B. thetaiotaomicron (Bt), B. fragilis (Bf), or B. vulgatus (Bvm) mouse isolate expressing the BT1491Δ25-NL fusion protein and feces was sampled on day 14 (Bt and Bf) or 8 (Bvm). 3% dextran sulfate sodium (DSS) and 25 mM IPTG was added in drinking water on day 7 (Bt and Bf) or day 4 (Bvm) where indicated. FIG. 12A is a graph showing the colony forming units (CFU)/mg feces counted after plating feces on selective media (BHIS+Gm+Em) for Bacteroides and plasmid selection. Shown are means and values of individual mice are represented by individual dots (n=5). ‘O’ indicate CFUs that exceeded the upper detection limit. Data for Bt and Bf was obtained in the same experiment and data for Bvm in a separate experiment. FIG. 12B shows luciferase activity in mice feces 8 days after gavage of B. vulgatus (Bvm) expressing the BT1491-NL fusion protein, as measured by relative light units per colony forming unit (RLU/CFU). Expression of the fusion protein was induced by 25 mM IPTG in the drinking water starting on day 4 where indicated. Shown are means and values of individual mice are represented by individual dots (n=5). Data was obtained from one experiment.

FIGS. 13A-13D show that colonization with Bacteroides spp. does not exacerbate intestinal inflammation in mice with DSS colitis. Colon length (FIGS. 13A-13B) and histological scores (FIGS. 13C-13D) of mice colonized with engineered B. thetaiotaomicron (Bt), B. fragilis (Bf) (FIGS. 13A, 13C), or B. vulgatus mouse isolate (Bvm) (FIGS. 13B, 13D) expressing the BT1491-NL fusion protein, or not colonized with engineered bacteria (no bacteria). Colitis was induced by dextran sulfate sodium (DSS) after Bacteroides colonization where indicated. Shown are means±SD and values of individual mice of one experiment with n=5. Data for Bt and Bf were obtained in the same experiment and data for Bvm in a separate experiment. ***P<0.001 and ****P<0.0001 as determined by unpaired t-test. ns, not significant.

### DETAILED DESCRIPTION

Microorganisms are essential for human health. The human body accommodates at least as many microbes—collectively referred to as the microbiota—as human cells (1-3). The intestinal mucosa harbors the largest population of microbial cells composed of an impressive variety of 500 to 1,000 different species adding up to an aggregate biomass of about 1.5 kg. Concentrations can exist up to 10¹¹ organisms per milliliter proximal colonic contents (2, 4). Although most members belong to the domain Bacteria, there are also viruses as well as Archaea and Fungi (5-7). Approximately 99.9% of all cultivatable bacteria are obligate anaerobes (8) with Bacteroides, Clostridium, Eubacterium, and Bifidobacterium (2, 9) as common genera. During the first years of live, every human develops a specific composition of microbes, which is continuously shaped by factors like diet, age, and antibiotics (10, 11).

The complex relationship between the gut flora and host can be commensal (i.e. benefitting from the host without affecting it), mutualistic, meaning that both, host and microbiota, benefit from each other, or pathogenic by harming the host (9, 12). Intestinal bacteria contribute to the hosts' health in many ways and are essential for its well-being. Indeed, the microbiota is sometimes referred to as the ‘forgotten organ’ to emphasize its crucial role in human health and disease (13). It endows us with functional features and metabolic pathways we have not evolved ourselves and are unable to perform. These include the fermentation of complex dietary carbohydrates supplying us with 10-15% of our daily calories (14, 15), the biotransformation of conjugated bile acids (16), and the synthesis of certain vitamins, particularly those of the B group and vitamin K, that are essential for human health (17). Moreover, the gut microbiota prevents intestinal colonization of potential pathogens and their translocation through the mucosa (18-20), and educates our immune system to induce tolerance to certain microbial epitopes, which contributes to reduce allergic responses to food and other environmental antigens (21, 22).

Recent efforts focused on the development of microbiota-derived therapeutics including genetically engineered probiotics that augment their intrinsic benefits by expressing recombinant therapeutic molecules (23, 24). As shown in the working examples, abundant species of the mutualistic Bacteroides genus were engineered to secrete therapeutic proteins associated with outer membrane vesicles (OMVs).

### Bacteroides Species Benefit the Human Host

Bacteroides are the predominant genus of the healthy human microbiota residing in the distal small intestine and colon (8, 25). They are a pleomorphic group of Gram-negative, obligate anaerobic, rod-shaped, non-spore-forming bacteria, which are essential for the mutualism between gut microbiota and human host. The Bacteroides spp. most abundant in the colon are B. vulgatus, B. thetaiotaomicron, and B. distasonis (ca. 10¹⁰ per g dry weight of feces) followed by B. fragilis, B. ovatus, and B. uniformis (ca. 10⁹ per g dry weight of feces) (26). B. fragilis and B. thetaiotaomicron are the most intensively studied species.

Accounting for ˜30% of the total bacterial population in the adult human gut (26), Bacteroides spp. live in an inextricable partnership with their host. They sense and adapt to environmental changes and stressors like altered nutrient availability or low oxygen concentrations, enabling them to thrive in the extremely harsh conditions in the gut (27). For instance, Bacteroides are nutritionally versatile in that they are able to use a wide range of carbon sources, including dietary fibers that are indigestible by the host (26). Lee et al. discovered that commensal colonization factors (ccf) important for glycan utilization are required for the persistent and resilient colonization of the mammalian gut by Bacteroides (28). Additionally, Bacteroides have multiple efflux pump systems to remove toxic substances (27, 29). As a consequence, they are significantly more stable members of the microbiota than the population average (30).

Protection from Disease.

Bacteroides spp. live in a mutually beneficial relationship with their host as long as they are retained within the intestine (4). However, if they escape the gut, usually as a consequence of intestinal surgery or ruptures, Bacteroides cause serious infections and abscesses at multiple body sites including the abdomen, liver, lungs, and brain, as well as severe bacteremia in rare cases (31). Toxigenic variants of B. fragilis are the most commonly encountered anaerobic pathogen and the most virulent Bacteroides species, even though B. fragilis accounts for only 0.5% of the human colonic microbiota (32).

Retained within the gut, Bacteroides substantially contribute to restrict pathogens from colonizing the intestine and invading host tissues by occupying ecological niches, nutrients, and other resources. In addition, B. fragilis makes a major contribution towards the development of the intestinal immune system (18, 33) to further limit the access and proliferation of potential pathogens into the gut. It prevents experimental intestinal inflammatory diseases in mice through a mechanism involving its capsular polysaccharide A (PSA) (34, 35). Further, B. thetaiotaomicron stimulates Paneth cells to produce antimicrobial peptides such as defensins and lectins (36). The secretion of angiogenin-4 in mouse Paneth cells, stimulated by B. thetaiotaomicron, has also been found to have bactericidal activity against certain intestinal Gram-positive pathogens like Listeria monocytogenes (37). Intriguingly, Bacteroides species were recently found to induce a distinct population of regulatory T cells (T_{reg}s) to confine immune-inflammatory responses (38). Monocolonization of germ-free mice with various Bacteroides species revealed increased frequency of these anti-inflammatory, beneficial RORγ⁺T_{reg}s, especially for B. ovatus, B. vulgatus, and B. thetaiotaomicron.

Nutrient Provision.

Bacteroides species have the remarkable ability to utilize a tremendous variability of nutrients. They ferment a large variety of indigestible dietary plant polysaccharides like amylose and amylopectin as well as host- or microbiota-derived polysaccharides that are not processed by human enzymes (14). They are responsible for the major fraction of polysaccharide digestion in the colon (26, 39), a task that is almost exclusively executed by members of the genus Bacteroides (40, 41). In particular, B. thetaiotaomicron plays an exceptional role in polysaccharide breakdown. The majority of its genome is devoted to an extensive polysaccharide utilization system that comprises 20 sugar-specific transporters, 163 homologs of polysaccharide-binding proteins (SusC and SusD homologs), and 172 glycosylhydrolases (e.g., glucosidases, galactosidases, mannosidases, amylases) (27). Notably, the number of glycosylhydrolases in the proteome of B. thetaiotaomicron is higher than in any other sequenced prokaryote (27). In addition, Bacteroides harbor multiple nutrient sensing mechanisms, including σ-factors and two-component regulatory systems that coordinate gene expression according to nutrient availability in the vicinity (27, 42). Consequently, Bacteroides are capable to adapt to changes and stresses in their environment.

By providing additional nutrients, Bacteroides spp. benefit the host as well as the whole bacterial community (43). Other organisms in the intestine that do not harbor such an array of sugar utilization enzymes can harvest sugars generated by Bacteroides. For example, B. ovatus ferments the fructose polymer inulin to cross-feed other gut species like B. vulgatus, which provide benefits for B. ovatus in return (44).

For these beneficial interactions, protein secretion plays a pivotal role to e.g. disseminate enzymes that make nutrients accessible for bystanders. Traditionally, six major classes of protein secretion machineries are known that translocate soluble molecules across the inner and outer membranes of Gram-negative bacteria (45). However, recent attention was drawn to a seventh, independent secretion system for the transfer of a diverse group of molecules via blebs formed by the outer membrane (46-48). These so called outer membrane vesicles (OMVs) are ubiquitously present in all Gram-negative bacteria (48-50) and possess many important advantages over other secretion systems, as elaborated on in the following two sections.

### Secretion of Bacterial Molecules by Outer Membrane Vesicles (OMVs)

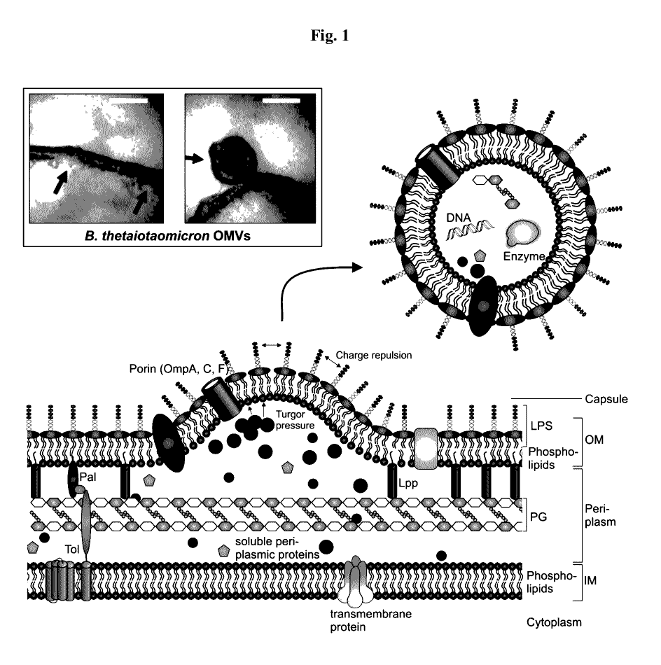

OMVs are spherical, bilayered proteoliposomes with a diameter ranging from 20 to 250 nm, consisting of the bacterial outer membrane and periplasmic content (FIG. 1) (46). They are constitutively shed from the outer membrane of Gram-negative bacteria during growth both in vitro and in vivo as well as in a variety of environments—from marine ecosystems over biofilms to mammalian hosts (51-53). As they pinch off from the cell surface, OMVs form from lipids and proteins embedded in the outer membrane and enclose soluble periplasmic components in their lumina. Secreted OMVs spread and deliver their cargo to distant sites, thus allowing the bacterium of origin to interact with their environment and eventually contributing to the fitness of the bacterium.

OMV Biogenesis.

OMVs originate from the cell envelope of Gram-negative bacteria, which is composed of two membranes, the outer membrane (OM) and the inner membrane (IM). The membranes are linked by a thin, mesh-like peptidoglycan (PG) network in the periplasmic space between the two and stitched together by protein crosslinks reaching from the IM through the PG network to the OM (FIG. 1). The inner membrane is a phospholipid bilayer, whereas the outer membrane comprises an interior leaflet of phospholipids and an exterior leaflet of glycolipids, principally lipopolysaccharides (LPS). Proteins integrated in the envelope can be either soluble proteins in the periplasm, transmembrane proteins, or lipoproteins that are anchored in the leaflet of either membrane via covalently attached lipid moieties (54).

The biogenesis of OMVs is an elaborate, energy-consuming mechanism that takes place during active growth and is not a by-product of cell lysis or a product of simple membrane shearing or blebbing (46). Vesiculation levels are induced by stress, for instance temperature increase (55, 56), amino acid deprivation (57), and antibiotics (58). However, the exact pathway of OMV formation remains unknown. Instead of a universal mechanism, the current literature proposes several mechanistic scenarios and key features that are likely to be involved (reviewed in detail in (59)).

In the first scenario, the OM bulges out in areas where it is dissociated from the underlying PG since protein crosslinks between the two are locally absent or decimated. Evidence for this model comes from hypervesiculating mutants of Escherichia coli that exhibit lower rates of OM-PG crosslinks than wild type E. coli (60). Further, proteins responsible for OM-PG crosslinks are sparse in OMVs; e. g. Braun's lipoprotein Lpp that covalently bridges the OM with the PG layer is excluded from E. coli OMVs (61). A second model of OMV biogenesis assumes vesiculation being a general stress response of bacteria to misfolded proteins or aberrant envelope components like overexpressed periplasmic proteins or excess peptidoglycan fragments (56, 62, 63). This material accumulates in so called nanoterritories at the inner surface of the OM, exerts turgor pressure on the OM and—after Lpp-PG crosslinks are locally removed—causes the OM to bulge outwards and bud off. Consequently, these undesired components are effectively removed from the cell and were found to be enriched in OMVs (56, 62, 64). The third theory assumes that altered biophysical characteristics of the OM change the membrane fluidity and flexibility, resulting in curvature and budding off (46, 48). For instance, charge-to-charge repulsion in microdomains of highly charged B-band LPS in Pseudomonas aeruginosa forces the membrane to curve outward and is enriched in OMVs (58, 65). Importantly, the suggested mechanisms are not mutually exclusive but rather may collectively contribute to the formation of OMVs (59, 66). In all cases, OMV biogenesis does not compromise envelope integrity (67).

Cargo Selection.

The composition of OMVs has been thoroughly analyzed by several groups in a multitude of bacterial strains. Mass spectrometry-based high-throughput profiling of OMVs has provided massive amounts of data about their protein content, which also elucidates their biogenesis and function (reviewed in (68)). Derived from the cell envelope, OMVs contain a similar outer membrane consisting of LPS and phospholipids as well as lipoproteins and membrane proteins like porins, ion channels, adhesins, and enzymes. Apart from periplasmic proteins and peptidoglycans, OMVs also carry specific cargos in their lumina, e. g. proteases, nucleic acids, and toxins such as the cholera toxin in Vibrio cholerae (69) and Cytolysin A in enterotoxic E. coli (70).

Although most proteins detected in OMVs were also found in the outer membrane or periplasm, certain proteins were specifically enriched or excluded in OMVs (61, 71-73). For instance, a proteomic study of the B. fragilis outer membrane and OMVs identified 40 proteins unique to OMVs, mostly glycosidases and proteases (74). This clustering of functionally related proteins supports the presence of a specific mechanism for OMV biogenesis, as discussed above, and additionally suggests a specialized machinery to sort certain proteins into OMVs. In line with this finding, also cytoplasmic and inner membrane proteins were identified in OMVs in low amounts, even after stringent purification steps (73). As these proteins are normally not present in the cell envelope, they must be actively sorted and exported into OMVs. Knowledge about how OMV cargo is selected would facilitate engineering of Gram-negative bacteria to specifically package heterologous proteins into their OMVs. However, the exact mechanism by which proteins are sorted into OMVs is currently not known, but several hypotheses exist (75).

In order to be secreted in OMVs, the cargo must be exported from the cytosol to the periplasm or OM first. Proteins in these two compartments are synthesized in the cytosol as precursors with N-terminal signal peptides. Typical amino acid motifs in the signal peptides target the proteins for translocation across the IM either by the Sec translocon (76-78) or the twin-arginine translocation (Tat) pathway (79, 80). After cleavage of the signal peptide at the periplasmic face of the IM, proteins destined for the OM cross the periplasm in complex with guiding chaperones (81, 82). To date, however, no signal or machinery has been identified to target incorporation of specific proteins into OMVs. One possibility is that OM components like OM proteins or OM lipoproteins prone to budding by increasing membrane curvature or fluidity might be inherently clustered in certain areas of the cell envelope during its biogenesis. Prior to OMV budding, specific proteins might directly or indirectly interact with the periplasmic face of these OM components and become enriched in OMVs (48). However, this model does not explain how cytoplasmic proteins that do not possess signal peptides are transported across the IM (83, 84) to interact with OM proteins and translocate into OMVs.

Cargo Delivery to Host Cells.

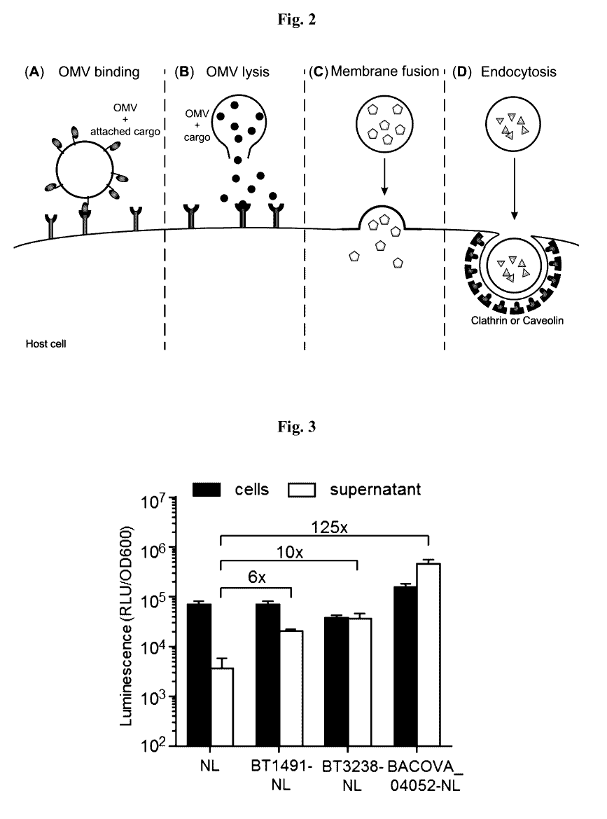

After disseminating, OMVs deliver their cargo not only to other bacterial cells (85, 86) but also eukaryotic host cells, for which three mechanisms were proposed (reviewed in (87)) (FIG. 2). First, OMVs may lyse or burst open in the proximity of target cells, releasing their content at high local concentrations at the effector site (47). Second, OMVs may attach to a target cell and deliver their content via fusion with the plasma membrane, despite the different architectures of bacterial OMV and eukaryotic cell membranes (88, 89). In cell culture, a fast cargo internalization was detected after 15 minutes of incubation with purified OMVs (70). Third, OMVs may enter non-phagocytic host cells as a whole entity via endocytic routes including macropinocytosis (90) and clathrin- or caveolin-dependent receptor-mediated endocytosis, e. g. via toll-like receptor 2 (91-95). Further, OMVs were observed to be engulfed by phagocytic host cells (49); for example in antigen-presenting cells (APCs) OMV phagocytosis induces the display of multiple bacterial epitopes on their surface. The three mechanisms were encountered in various bacterial species and in some cases more than one of the uptake routes was identified in the same species. Differences in OMV size and composition may favor a specific uptake route for optimal delivery and processing within the host cell (96).

### Functional Roles of OMVs in Physiology and Pathogenesis

As vesiculation is a ubiquitous mechanism in Gram-negative bacteria, it is obvious that OMVs play an integral role in cell physiology and the pathogenesis of infections (97). Depending on the species of origin and their environment, OMVs have diverse functions. In general, they act as long distance delivery vehicles of proteins, lipids, and genetic material from bacteria to bacteria or host cells while protecting their cargo form dilution and proteolytic degradation. OMVs induce changes in the bacterial environment and benefit the survival of the parent bacteria, as illustrated in the following section.

Initially, OMVs were thought to primarily mediate pathogenic processes by supporting the shuttle of virulence factors such as proteases, toxins, or pro-inflammatory molecules like flagellin, LPS, and peptidoglycan, to host cells and competing bacteria (58, 89). However, recent attention has been drawn towards non-pathogenic, commensal bacteria utilizing OMVs to mediate beneficial effects on the host (97-99).

OMVs Benefit the Bacterial Community.

A major role of OMVs in bacterial physiology lies in the response to environmental stress. Here, OMVs are an effective mechanism to quickly relieve the cell of damaging agents such as toxic or misfolded material, antibiotics, and bacteriophages and are particularly crucial for aggregates that are too big for OM pores. For instance, heat stress in E. coli results in the accumulation of misfolded proteins, which are packed into OMVs and thereby removed (56). OMVs may even be essential in the survival of stress situations. McBroom and Kuehn (56) found that when two vesiculation mutant E. coli strains were challenged with lethal envelope stressors, e.g. ethanol or OM-damaging antimicrobial peptides, hypovesiculating mutants succumbed, whereas hypervesiculating mutants survived better than wild type E. coli. Further, bacterial cells exposed to antibiotics produce OMVs to sequester (100) or degrade (101) the antibiotics outside of the cells. Additionally, increased OMV production was shown to protect bacteria from lytic bacteriophages by acting as ‘decoy’ targets for the phages (100, 102). Hence, Manning & Kuehn called OMVs an ‘innate bacterial defense’ (100).

Moreover, OMVs are essential in nutrient acquisition. They can carry and disseminate enzymes that degrade complex macromolecules to make nutrients accessible for bacterial and host cells. For instance, proteomic data revealed that B. fragilis and B. thetaiotaomicron preferentially target acidic hydrolytic enzymes, primarily proteases and glycosidases, to OMVs to help secure nutrients (74). Besides, OMVs can contain iron and zinc acquisition systems to collect these scarce metal ions from the environment and enrich them for the subsequent consumption by bacteria (73).

Importantly, OMVs act as a common resource that benefits whole bacterial populations: They not only provide nutrients for the OMV producing bacterium but also for bystanders. Also, OMVs were found to protect both producing and bystander bacteria from antibiotic stress by sequestration of antibiotics. Consequently, OMVs have an indispensable role for the survival and fitness of whole bacterial communities present in the gut microbiota (103).

OMVs Contribute to Host Health.

Apart from benefitting bacterial populations, OMVs also directly improve the human gastrointestinal physiology. For instance, Stentz et al. found that BtMinpp, a homolog of the mammalian Inositol hexakisphosphate (InsP₆) phosphatase (MINPP), is secreted in OMVs by B. thetaiotaomicron and delivered to intestinal epithelial cells (99). BtMinpp-packed OMVs thereby not only contribute to the essential InsP₆ homeostasis and free up the vital nutrients phosphate and inositol but also interact with the inositol polyphosphate signaling pathway in host cells. In addition, OMVs released by B. fragilis deliver immunomodulatory molecules to host cells (98). The capsular polysaccharide A (PSA) is selectively associated with B. fragilis OMVs, which are then internalized by dendritic cells (DCs) to program them for an enhanced production of T_{reg}s that secrete the anti-inflammatory cytokine IL-10. This leads to mucosal tolerance and protects mice from experimental colitis. These examples show two mechanisms of a beneficial inter-kingdom communication between microbiota and host mediated by OMVs.

### Advantages of OMV-Based Protein Secretion and Delivery

OMVs act as a secretion and delivery system to disseminate bacterial products to distant locations. As compared to whole cells, OMVs are smaller and more mobile, which enables them to reach remote sites and sites inaccessible to bacteria without consuming energy to move themselves (46). In contrast to traditional soluble secretion machineries, OMVs exhibit several advantages. Recently, Hickey et al. found that B. thetaiotaomicron OMVs can access host immune cells in the murine intestinal mucosa (104). Sulfatases contained in these OMVs enabled them to break through the sulfate-containing, net-like mucus layer, cross the epithelial barrier, and deliver their cargo after being engulfed by macrophages.

Unlike soluble secretory pathways, the secretion via OMVs protects cargo from degradation and dilution. Luminal OMV proteins resist proteolytic degradation, e.g. in the GI tract (105), allowing even less stable proteins to reach their destination. Further, OMVs are robust as they show no signs of spontaneous lysis and increased thermal stability (106). Sequestration in the enclosed OMV prevents cargo dilution and enables its delivery and release at high local concentrations over long distances. Due to the co-transport of multiple molecules, for instance the various enzymes required for the degradation of a complex molecule, they reach distant targets simultaneously, which increases their efficacy. Another advantage of OMV-based secretion is to efficiently shed insoluble hydrophobic molecules like lipids, membrane proteins, and certain signaling molecules.

Inflammatory Bowel Diseases and IL-10.

Inflammatory bowel disease (IBD) is a chronic, relapsing, intestinal disorder, frequently manifesting as Crohn's disease (CD) or ulcerative colitis (UC). The diseases are characterized by chronic inflammation, severe diarrhea with rectal bleeding, and malabsorption as a consequence of a dysregulated intestinal immune homeostasis (107). Although the causes are not fully elucidated, disproportionate mucosal immune responses against resident bacteria are thought to be crucially involved and might be fostered by both genetic and environmental factors (108). Innate and adaptive immune cells accumulate in the intestinal mucosa leading to increased levels of pro-inflammatory cytokines like IFN-γ, interleukin (IL)-17, and IL-22 produced by the T helper (Th)1 response in CD, and tumor necrosis factor (TNF)-α, IL-1β, and IL-6 mediated by the Th2-like response in UC (109). The chronic intestinal inflammation results in continuous epithelial damage and destruction of the epithelial barrier, which allows more intestinal microbes to invade and evoke further immune responses (110). CD and UC differ in the localization of the inflammation. While CD can affect any part of the GI tract, it is predominantly found in the terminal ileum with transmural inflammation across the entire intestinal wall. In contrast, UC is restricted to mucosal inflammation of the colon and rectum (111).

Genome-wide association studies have identified IL10 as a susceptibility locus for the development of IBD (112-114). Polymorphisms in the IL10 promoter that reduce serum levels of the anti-inflammatory cytokine IL-10 have been linked to certain forms of IBD (115, 116). Thus, IL-10 supplementation has been regarded as an alternative IBD treatment to the current available options like surgery, aminosalicylates, immunosuppressants, and biologics, which often have low response rates (117). The dose-limiting side effects for these long-term drug treatments range from nausea and headache to severe, long-lasting complications like osteoporosis or bone marrow toxicity resulting in leucopenia and sepsis (118, 119). Therefore, improved treatment options are urgently needed.

The multifunctional anti-inflammatory cytokine IL-10 counteracts excessive inflammatory immune responses and prevents tremendous intestinal damage. It is produced by many cell types of the innate (e.g. dendritic cells (DCs), macrophages, and natural killer (NK) cells) and adaptive immune system (e.g. Th1, Th2, T_{reg}s, CD8⁺ T cells, and B cells) (120). IL-10 binds its receptor as a homodimer, which is present on most hematopoietic cells and induces a downstream signaling cascade leading to signal transducer and activator of transcription 3 (STAT3) mediated gene expression (121, 122). As a consequence, IL-10 exerts a wide range of immunomodulatory effects. In macrophages, IL-10 inhibits antigen presentation by MHC class II, co-stimulatory molecule expression, and pro-inflammatory cytokine (e.g. TNF-α, IFN-γ) and chemokine production (reviewed in (120)). Further, Th1, Th2, and NK cell responses are inhibited and the differentiation of IL-10 producing T_{reg}s enhanced.

IL-10 supplementation alleviated symptoms in IBD animal models. Intestinal inflammation in several models of experimental colitis were substantially improved by IL-10 treatment in various animals including mice, rats, and rabbits (123-125). However, clinical studies indicate no significantly reduced remission rates or clinical improvements of systemic IL-10 therapy compared to placebo (118, 126). A hypothesis explaining this setback is that local IL-10 concentrations in the intestine were too low to elicit an ameliorating effect. Unfortunately, concentrations of systemically administered IL-10 are limited due to side effects like anemia and headache. To increase the mucosal bioavailability of IL-10 and circumvent systemic side effects, Steidler et al. engineered Lactococcus lactis to secrete IL-10 after intragastric administration (127). Dextran sulfate sodium (DSS) induced colitis was reduced by 50% in mice as determined by histological scores. Further, a small phase I human trial revealed that application of IL-10 producing L. lactis is safe and well-tolerated in humans while systemic side effects are avoided (128). These results show that IL-10 should have therapeutic potential in the treatment of intestinal inflammation if delivered at high concentrations to the diseased mucosa.

### Advantages of Engineered Bacteroides for Intestinal Drug Delivery

Genetically modified bacteria residing in the intestine and secreting therapeutic proteins in situ have crucial advantages over currently available systemic treatments. First, the drug is exclusively produced and released at the desired site. Hence, vital organs are protected from elevated drug doses that lead to toxicity and side effects. Further, it obviates the need to protect unstable drugs like therapeutic proteins from degradation on a long route to the effector site. Second, due to the targeted action of in vivo production and delivery, the required dosage for a comparable therapeutic effect is reduced by several orders of magnitudes (127, 129, 130), which additionally prevents side effects. Third, engineered bacteria can be orally administered and reach the intestine where they release the therapeutic proteins. This increases patient compliance compared to systemic treatment by invasive and inconvenient intravenous or subcutaneous injections. Finally, the treatment costs are drastically reduced as the need for expensive drug purification and formulation is eliminated.

Commensal Bacteroides colonizes the intestine naturally in high abundance providing a high capacity and continuity of drug production. As has been elaborated on in section 2.1., Bacteroides additionally have beneficial features for the host. The group of Simon Carding engineered B. ovatus to produce promising candidates for the long-term treatment of inflammatory gut diseases like transforming growth factor(TGF)-β, keratinocyte growth factor(KGF)-2, and IL-2 (129, 130, 134). The growth factors/cytokines were secreted into the extracellular milieu by B. ovatus and needed to diffuse to their target site in the mucosa. Provided herein is a model where therapeutic proteins are secreted in association with outer membrane vesicles produced by Bacteroides species and hence reach their target cells in a more directed and concentrated form.

### Applications

Deploying OMVs as drug delivery vehicles for therapeutic proteins produced by engineered bacteria will have several advantages compared to soluble secreted proteins. OMVs can migrate to the inflammatory site in the mucosa and deliver anti-inflammatory molecules directly to the target side. In contrast to whole bacteria migrating to the inflammatory site, OMVs are less immunogenic and therefore less prone to exacerbate the inflammation. Bacteroides OMVs are particularly suited as it was shown that B. fragilis LPS is 10 to 1,000 times less toxic than that of E. coli (31). Another benefit of OMV-based delivery is that multiple different therapeutic proteins can be targeted to the same OMVs and simultaneously be delivered to the same target cell. For instance, IBD could be treated as a combination therapy of various anti-inflammatory proteins that complement or reinforce each other, such as IL-10, IL-22, IL-4, and TGF-β (171).

Since OMV tag-mediated secretion is compatible with at least three different Bacteroides spp., therapeutic protein concentrations could be increased if necessary by employing several species.

As alternative to administration of engineered bacteria that secrete therapeutic proteins via OMVs, purified OMVs packed with protein drugs could be administered without the associated bacteria. Remarkably, Shen et al. showed that dosing of purified B. fragilis OMVs alone were sufficient to protect mice from chemically-induced colitis (172). By this approach, possible environmental and safety concerns raised by applying genetically modified organisms in patients will be overcome while still exploiting the benefits of targeted OMV drug delivery.

The OMV-based secretion system in Bacteroides spp. should substantially improve the quality of life of IBD patients. After a single administration of the engineered bacteria, they will reside in the patient's intestine and secrete therapeutic proteins. By coupling the production of therapeutic proteins to a sensing system for IBD flare-ups, the therapy is adjusted to the disease state in the patient. For instance, the infiltration of neutrophils during intestinal inflammation leads to a release of reactive oxygen species and increased oxygen levels in the gut (173). Additionally, nitric oxide (NO) concentrations were found to correlate with disease activity in ulcerative colitis with 100 times higher levels in the patients than control levels (174, 175). Therefore, oxidative stress pathways or NO-induced gene expression systems could be deployed for that purpose. Eventually, IBD patients will be colonized with engineered Bacteroides spp. that sense and react to disease flare-ups with the secretion of anti-inflammatory proteins directly at the inflamed site, reducing patient exposure to the drug to a minimum. Ideally, the inflammation will be alleviated before the patient suffers from the symptoms. As Bacteroides colonizes the intestine stably and in high abundance, it is better suited for these long-term application than L. lactis or E. coli. Additionally, the anaerobic nature of Bacteroides spp. provides an inherent biosafety feature.

Considering the 2.2 million Europeans and 1.4 million Americans suffering from IBD (176) and the dramatic increase of IBD occurrence in western countries as well as newly industrialized countries in Asia, South America, and the Middle East (177), there is an urgent need for novel effective treatments. Current therapy options like aminosalicylates, immunosuppressants, and biologics either show severe dose-limiting side effects including long-lasting complications like osteoporosis or bone marrow toxicity (118, 119), or have low response rates. Although the highly cost-intensive therapy with anti-TNF antibodies is currently seen as the gold-standard for IBD treatment, ca. 30% of patients to not respond and another 40% lose response over time (117).

Engineered commensal bacteria of the Bacteroides spp. secreting immunomodulatory molecules, such as IL-10, via OMVs, as described herein, should provide a specific and controlled long-term immune therapy for intestinal disorders such as IBD.

### Engineered Bacteroides

Some aspects of the present disclosure relate to engineered Bacteroides. Examples of species of Bacteroides that may be used in accordance with the present disclosure include, without limitation, B. acidifaciens, B. caccae, B. distasonis, B. gracilis, B. fragilis, B. dorei, B. oris, B. ovatus, B. putredinis, B. pyogenes, B. stercoris, B. suis, B. tectus, B. thetaiotaomicron, B. vulgatus, B. eggerthii, B. merdae, B. stercori, and B. uniformis.

In some embodiments, an engineered Bacteroides comprises a nucleic acid encoding a fusion protein comprising a Bacteroides membrane-associated protein linked to a heterologous protein (e.g., a therapeutic protein). In some embodiments, a promoter is an inducible promoter. In some embodiments, an engineered Bacteroides comprises a fusion protein comprising a Bacteroides membrane-associated protein linked to a heterologous protein (e.g., a therapeutic protein). A “fusion protein” is a hybrid polypeptide that comprises protein domains (e.g., at least one peptide) from at least two different proteins (e.g., obtained from two different types of proteins). In some embodiments, the Bacteroides membrane-associated protein may be fused to the N-terminus of the heterologous protein. In other embodiments, the Bacteroides membrane-associated protein may be fused to the C-terminus of the heterologous protein.

A “membrane-associated protein” is a protein, truncated protein or peptide that interacts with, or is part of, a cell membrane (e.g., a lipid bilayer). Non-limiting examples of membrane-associated proteins include integral membrane proteins (e.g., that are permanently anchored or part of the membrane) and peripheral membrane proteins (e.g., that are only temporarily attached to the lipid bilayer or to other integral proteins). In some embodiments, a Bacteroides membrane-associated protein is a Bacteroides lipoprotein. Bacteroides lipoproteins include membrane proteins that play key roles in Bacteroides physiology and pathogenesis, e.g., in host cell adhesion, modulation of inflammatory processes, and translocation of proteins, e.g., virulence factors, into host cells. A lipoprotein may be or comprise a signal peptidase I (SPI) or a signal peptidase II (SPII) lipoprotein. In some embodiments, a lipoprotein does not include a signal peptidase.

In some embodiments, a Bacteroides membrane-associated protein is selected from the group consisting of: BT1491 proteins, BT3238 proteins, and BACOVA_04502 proteins. In some embodiments, a Bacteroides membrane-associated protein is a truncated variant of a BT1491 protein, a BT3238 protein, or a BACOVA_04502 protein. For example, a Bacteroides membrane-associated protein may be a N-terminal peptide of a BT1491 protein, a BT3238 protein, or a BACOVA_04502 protein. In some embodiments, a N-terminal peptide of a BT1491 protein, a BT3238 protein, or a BACOVA_04502 protein has a length of 18-100 amino acids. For example, a N-terminal peptide of a BT1491 protein, a BT3238 protein, or a BACOVA_04502 protein may have a length of 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 89, 90, 91, 92, 93, 94, 95, 96, 97, 98, 99, or 100 amino acids. In some embodiments, a N-terminal peptide of a BT1491 protein, a BT3238 protein, or a BACOVA_04502 protein has a length of 25-55 amino acids. In some embodiments, a N-terminal peptide of a BT1491 protein, a BT3238 protein, or a BACOVA_04502 protein has a length of 18-50 amino acids. In some embodiments, a N-terminal peptide of a BT1491 protein has a length of 25 amino acids. In some embodiments, a N-terminal peptide of a BT3238 protein has a length of 35 amino acids. In some embodiments, a N-terminal peptide of a BACOVA_04502 protein has a length of 28 amino acids.

In some embodiments, a Bacteroides lipoprotein of the present disclosure comprises a N-terminal signal peptide. A “signal peptide” is a peptide located within the N-terminal region (e.g., 15-60 amino acids) of a protein. A signal peptide, in some instances, is needed for translocation across a cell membrane and thus universally controls in eukaryotes and prokaryotes entry of most proteins into the secretory pathway. A signal peptides generally includes three regions: an N-terminal region of differing length, which usually comprises positively charged amino acids; a hydrophobic region; and a short carboxy-terminal peptide region. Bacteroides lipoproteins of the present disclosure, in some embodiments, comprises a N-terminal signal peptide that is rich in aspartic acids (D). For example, the N-terminal signal peptide may comprise at least 40%, at least 50%, at least 60%, at least 70%, at least 80%, or more aspartic acids. In some embodiments, a N-terminal signal peptide comprises 30%, 35%, 40%, 45%, 50%, 55%, 60%, 65%, 70%, 75%, 80%, or more aspartic acids.

In some embodiments, a N-terminal signal peptide comprises a cleavage site for a signal peptidase, e.g., a signal peptidase I (SPI) or a signal peptidase II (SPIT). In some embodiments, a signal peptidase cleavage site is within the first 15-50 amino acids of the Bacteroides lipoprotein. For example, a signal peptidase cleavage site may be within the first 15, 20, 25, 30, 35, 40, 45, or 50 amino acids of a Bacteroides lipoprotein.

A fusion protein of the disclosure comprises Bacteroides membrane-associate protein linked to a heterologous protein. A “heterologous protein,” generally, is any non-Bacteroides protein. Non-limiting examples of heterologous protein include recombinant therapeutic proteins, diagnostic proteins and prophylactic proteins. In some embodiments, however, a Bacteroides membrane-associate protein may be linked to a Bacteroides protein. Specific examples of heterologous proteins include, without limitation, biomarkers, transcriptional regulators, epigenetic modifiers, nucleic acid editing enzymes, nucleases, proteases, or any other enzymes of interest.

In some embodiments, a heterologous protein is a therapeutic protein. Therapeutic proteins that may be used in accordance with the present disclosure include, without limitation, antibodies, cytokines, and growth factors. In some embodiments, the therapeutic protein is a growth factor, e.g., a transforming growth factor beta 1 (TGF-β1) or a keratinocyte growth factor (KGF). In some embodiments, a therapeutic protein is a cytokine. Cytokines include small cell-signaling protein molecules secreted by cells. Non-limiting examples of cytokines that may be used in accordance with the present disclosure include Acrp30, AgRP, amphiregulin, angiopoietin-1, AXL, BDNF, bFGF, BLC, BMP-4, BMP-6, b-NGF, BTC, CCL28, Ck beta 8-1, CNTF, CTACK CTAC, Skinkine, Dtk, EGF, EGF-R, ENA-78, eotaxin, eotaxin-2, MPIF-2, eotaxin-3, MIP-4-alpha, Fas, Fas/TNFRSF6/Apo-1/CD95, FGF-4, FGF-6, FGF-7, FGF-9, Flt-3 Ligand fms-like tyrosine kinase-3, FKN or FK, GCP-2, GCSF, GDNF Glial, GITR, GITR, GM-CSF, GRO, GRO-α, HCC-4, hematopoietic growth factor, hepatocyte growth factor, 1-309, ICAM-1, ICAM-3, IFN-γ, IGFBP-1, IGFBP-2, IGFBP-3, IGFBP-4, IGFBP-6, IGF-I, IGF-I SR, IL-1α, IL-1β, IL-1, IL-1 R4, ST2, IL-3, IL-4, IL-5, IL-6, IL-8, IL-10, IL-11, IL-12 p40, IL-12p70, IL-13, IL-16, IL-17, I-TAC, alpha chemoattractant, lymphotactin, MCP-1, MCP-2, MCP-3, MCP-4, M-CSF, MDC, MIF, MIG, MIP-1α, MIP-1β, MIP-1δ, MIP-3α, MIP-3β, MSP-a, NAP-2, NT-3, NT-4, osteoprotegerin, oncostatin M, PARC, PDGF, P1GF, RANTES, SCF, SDF-1, soluble glycoprotein 130, soluble TNF receptor I, soluble TNF receptor II, TARC, TECK, TGF-beta 1, TGF-beta 3, TIMP-1, TIMP-2, TNF-α, TNF-β, thrombopoietin, TRAIL R3, TRAIL R4, uPAR, VEGF and VEGF-D. In some embodiments, a cytokine is interleukin 2 (IL-2), interleukin 10 (IL-10), or interleukin 22 (IL-22). In some embodiments, a therapeutic protein is interleukin 10 (IL-10) or a functional fragment thereof.

In some embodiments, a Bacteroides membrane-associated protein facilitates the secretion of a fusion protein into the periplasm of the engineered Bacteroides. In some embodiments, a Bacteroides membrane-associated protein facilitates the display of the fusion protein on the outer membrane of the engineered Bacteroides. In some embodiments, a fusion protein is incorporated into a Bacteroides outer membrane vesicle (OMV).

Accordingly, some aspects of the present disclosure provides engineered Bacteroides outer membrane vesicles (OMVs) comprising a fusion protein, as described herein. A Bacteroides OMV refers to a spherical bud of the outer membrane filled with outer membrane and periplasmic contents. OMVs are commonly produced by Gram-negative bacteria. The production of OMVs allows bacteria to interact with their environment, and OMVs have been found to mediate diverse functions, including promoting pathogenesis, enabling bacterial survival during stress conditions and regulating microbial interactions within bacterial communities. Derived from the cell envelope, OMVs contain a similar outer membrane consisting of LPS and phospholipids as well as lipoproteins and membrane proteins like porins, ion channels, adhesins, and enzymes. Apart from periplasmic proteins and peptidoglycans, OMVs also carry specific cargos in their lumina, e.g., proteases, nucleic acids, and toxins such as the cholera toxin in Vibrio cholerae and Cytolysin A in enterotoxic E. coli. In some embodiments, a fusion protein of the present disclosure, when incorporated into the engineered Bacteroides OMV, is in the lumen of the OMV, or is displayed on the surface of the OMV.

In some embodiments, an engineered Bacteroides OMV of the present disclosure maybe used to deliver a fusion protein to another cell, e.g., a eukaryotic cell. In some embodiments, a fusion protein is delivered to an immune cell. For example, a fusion protein may be delivered to a B cell, a dendritic cell, a granulocyte, a megakaryocyte, a monocytes/macrophage, a natural killer cell, a platelet, a red blood cell, or a T cell or a thymocyte. In some embodiments, an immune cell is an intestinal mucosal immune cell. An intestinal mucosal immune cell is a component of the mucosal immune system at the gastrointestinal barrier, which contains small foci of lymphocytes and plasma cells are scattered widely throughout the lamina propria of the gut wall. One skilled in the art is familiar with different types of immune cells and the gastrointestinal mucosal immune system.

To deliver the fusion protein to another cell, an engineered Bacteroides OMV interacts with the cell, e.g., the immune cell. Fusion proteins may be delivered in a number of different ways. For example, in some embodiments, a fusion protein is displayed on the surface of an engineered Bacteroides OMV and is recognized by a receptor on the surface of a cell, e.g., an immune cell, receiving the fusion protein. In some embodiments, the engineered Bacteroides OMV undergoes lysis and releases the fusion protein to the vicinity of the cell receiving the fusion protein. In some embodiments, an engineered Bacteroides OMV undergoes membrane fusion with the cell receiving the fusion protein. In some embodiments, an engineered Bacteroides OMV is internalized as a whole entity by the cell receiving the fusion protein via endocytosis.

An engineered Bacteroides or engineered Bacteroides OMV of the present disclosure, may be administered to a subject. In some embodiments, a subject has a disorder that may be treated with a heterologous protein delivered by an OMV of an engineered Bacteroides. In some embodiments, the disorder is an intestinal disorder. For example, an intestinal disorder may be inflammatory bowel disease (IBD) or Crohn's disease. In some embodiments, an engineered Bacteroides or engineered Bacteroides OMV is administered orally or intrarectally.

In some embodiments, a subject is a mammal. In some embodiments, a subject is human.

A “nucleic acid” refers to at least two nucleotides covalently linked together, and in some instances, may contain phosphodiester bonds (e.g., a phosphodiester “backbone”). In some embodiments, a nucleic acid (e.g., an engineered nucleic acid) of the present disclosure may be considered a nucleic acid analog, which may contain other backbones comprising, for example, phosphoramide, phosphorothioate, phosphorodithioate, O-methylphophoroamidite linkages, and/or peptide nucleic acids. Nucleic acids (e.g., components, or portions, of the nucleic acids) of the present disclosure may be naturally occurring or engineered. Nucleic acids of the present disclosure may be single-stranded (ss) or double-stranded (ds), as specified, or may contain portions of both single-stranded and double-stranded sequence (e.g., a single-stranded nucleic acid with stem-loop structures may be considered to contain both single-stranded and double-stranded sequence). It should be understood that a double-stranded nucleic acid is formed by hybridization of two single-stranded nucleic acids to each other. Nucleic acids may be DNA, including genomic DNA and cDNA, RNA or a hybrid/chimeric of any two or more of the foregoing, where the nucleic acid contains any combination of deoxyribo- and ribonucleotides, and any combination of bases, including uracil, adenine, thymine, cytosine, guanine, inosine, xanthine, hypoxanthine, isocytosine, and isoguanine.

An “engineered nucleic acid” is a nucleic acid that does not occur in nature. It should be understood, however, that while an engineered nucleic acid as a whole is not naturally-occurring, it may include nucleotide sequences that occur in nature. In some embodiments, an engineered nucleic acid comprises nucleotide sequences from different organisms (e.g., from different species). For example, in some embodiments, an engineered nucleic acid includes a murine nucleotide sequence, a bacterial nucleotide sequence, a human nucleotide sequence, and/or a viral nucleotide sequence. The term “engineered nucleic acids” includes recombinant nucleic acids and synthetic nucleic acids. A “recombinant nucleic acid” refers to a molecule that is constructed by joining nucleic acid molecules and, in some embodiments, can replicate in a live cell. A “synthetic nucleic acid” refers to a molecule that is amplified or chemically, or by other means, synthesized. Synthetic nucleic acids include those that are chemically modified, or otherwise modified, but can base pair with naturally-occurring nucleic acid molecules. Recombinant nucleic acids and synthetic nucleic acids also include those molecules that result from the replication of either of the foregoing. Engineered nucleic acids of the present disclosure may be encoded by a single molecule (e.g., included in the same plasmid or other vector) or by multiple different molecules (e.g., multiple different independently-replicating molecules).

Engineered nucleic acids of the present disclosure may be produced using standard molecular biology methods (see, e.g., Green and Sambrook, Molecular Cloning, A Laboratory Manual, 2012, Cold Spring Harbor Press). In some embodiments, engineered nucleic acids are produced using GIBSON ASSEMBLY® Cloning (see, e.g., Gibson, D. G. et al. Nature Methods, 343-345, 2009; and Gibson, D. G. et al. Nature Methods, 901-903, 2010, each of which is incorporated by reference herein). GIBSON ASSEMBLY® typically uses three enzymatic activities in a single-tube reaction: 5′ exonuclease, the Y extension activity of a DNA polymerase and DNA ligase activity. The 5′ exonuclease activity chews back the 5′ end sequences and exposes the complementary sequence for annealing. The polymerase activity then fills in the gaps on the annealed regions. A DNA ligase then seals the nick and covalently links the DNA fragments together. The overlapping sequence of adjoining fragments is much longer than those used in Golden Gate Assembly, and therefore results in a higher percentage of correct assemblies.

Engineered nucleic acids of the present disclosure may be included within a vector, for example, for delivery to a cell. A “vector” refers to a nucleic acid (e.g., DNA) used as a vehicle to artificially carry genetic material (e.g., an engineered nucleic acid construct) into a cell where, for example, it can be replicated and/or expressed. In some embodiments, a vector is an episomal vector (see, e.g., Van Craenenbroeck K. et al. Eur. J. Biochem. 261, 5665, 2000, incorporated by reference herein). A non-limiting example of a vector is a plasmid. Plasmids are double-stranded generally circular DNA sequences that are capable of automatically replicating in a host cell. Plasmid vectors typically contain an origin of replication that allows for semi-independent replication of the plasmid in the host and also the transgene insert. Plasmids may have more features, including, for example, a “multiple cloning site,” which includes nucleotide overhangs for insertion of a nucleic acid insert, and multiple restriction enzyme consensus sites to either side of the insert. Another non-limiting example of a vector is a viral vector. Any of the engineered nucleic acids of the present disclosure, for example, a nucleic acid encoding a fusion protein, may be present on a vector (e.g., and delivered to a Bacteroides cell).

A “promoter” refers to a control region of a nucleic acid sequence at which initiation and rate of transcription of the remainder of a nucleic acid sequence are controlled. A promoter may also contain sub-regions at which regulatory proteins and molecules may bind, such as RNA polymerase and other transcription factors. Promoters may be constitutive, inducible, activatable, repressible, tissue-specific or any combination thereof. A promoter drives expression or drives transcription of the nucleic acid sequence that it regulates. A promoter is considered to be “operably linked” when it is in a correct functional location and orientation in relation to a nucleic acid sequence it regulates to control (“drive”) transcriptional initiation and/or expression of that sequence.

Promoters of an engineered nucleic acid construct may be “inducible promoters,” which refer to promoters that are characterized by regulating (e.g., initiating or activating) transcriptional activity when in the presence of, influenced by or contacted by an inducer signal.

### Examples

Effective treatment of intestinal disorders using protein drugs such as anti-inflammatory cytokines is hampered by too low therapeutic levels at the required site—the intestinal mucosa; oral administration is impeded by protein degradation in the acid-rich and protease-rich upper gastro-intestinal tract; and systemic medication leads to side effects and requires frequent injections of high doses due to the short in vivo half-lives of proteins. One approach to overcome these difficulties is targeted enteric protein delivery by genetically engineered bacteria, as provided herein. Abundant, commensal Bacteroides species naturally and stably colonize the gut in concentrations of up to 30% of the intestinal flora.

In the present study, Bacteroides spp. was engineered to produce therapeutic proteins for intestinal delivery. The proteins were packaged into outer membrane vesicles (OMVs), which are constitutively produced by all Gram-negative bacteria. The circumventing membrane of OMVs protects their cargo from dilution and proteolytic degradation by intestinal proteases even when transported over long distances. Surprisingly, Bacteroides OMVs were found to cross the epithelial layer and deliver their cargo to host immune cells (104). This was harnessed by packing anti-inflammatory molecules such as interleukin-10 into Bacteroides OMVs, which deliver the molecules to mucosal immune cells, leading to amelioration of intestinal inflammation.

Heterologous proteins were targeted to OMVs by genetic fusion to peptide tags derived from Bacteroides OMV proteins. Three proteins enriched in OMVs were first identified by literature research and whether fusion to a reporter protein leads to its integration and activity in OMVs was tested. To prohibit disturbing effects due to the bulky OMV proteins, the minimal sequence tags crucial for the export to OMVs were determined. The anti-inflammatory cytokine IL-10 was then targeted to OMVs for future usage in the treatment of inflammatory bowel diseases (IBD). Additionally, the colonization behavior and tolerance of different Bacteroides spp. in the mouse gut were analyzed to prepare for subsequent in vivo tests of the designed fusion proteins delivered by the optimal chassis.

Thus, provided herein are treatment systems used for intestinal protein delivery that require low dosage due to targeted high concentrations, are cost-effective, are well-tolerated, and are potentially applicable to many diseases. The methods of the present disclosure are suitable for a wide range of therapeutic proteins that currently encounter difficulties with regard to intestinal delivery.

To develop a versatile and effective intestinal protein delivery system by harnessing a human gut commensal, Bacteroides were engineered to produce outer membrane vesicles (OMVs) that carry heterologous proteins which can be delivered to host cells in the gut—once applied to humans. Bacteroides spp. are one of the numerically dominant beneficial genera of the human intestinal microbiota with stable and robust colonization (28, 30), which particularly qualifies them for long-term therapeutics. Recently, the genetic parts for the precise modulation of gene expression in B. thetaiotaomicron were designed by our group (140). The isopropyl β-D-1-thiogalactopyranoside (IPTG)-inducible expression system, which integrates into the Bacteroides genome after conjugation from E. coli, was deployed. The present study to generate therapeutic Bacteroides spp. was conducted in a two-part process. In the first part, for suitable fusion tags derived from proteins in B. thetaiotaomicron and B. ovatus OMVs, which are able to target heterologous proteins to OMVs of Bacteroides spp., were examined. In the second part, the in vivo tolerance and colonization behavior of different Bacteroides species were tested in a mouse model, identifying exemplary species for use in intestinal delivery of the fusion proteins.

### Example 1: Identification of Protein Fusion Tags for OMV Targeting

While mass spectrometry and biochemical analyses have shown that some proteins are enriched or exclusively found in OMVs, including those generated by Bacteroides spp. (74), the mechanism of their selection remains elusive. At present, it is unknown if a particular part of an OMV protein is responsible for its secretion via Bacteroides OMVs. This question was addressed through the investigation of whether the fusion of heterologous proteins to native OMV proteins promotes their targeting to OMVs and if truncated peptide tags derived from the OMV proteins' N-termini suffice in doing so.

The OMV Proteins BT1491, BT3238, and BACOVA_04502 Promote Enrichment of Heterologous Proteins in OMV-Containing Culture Supernatants.

First, the literature for Bacteroides proteins found enriched in OMVs was searched in order to identify possible candidates for OMV targeting. In the only large-scale proteomic analysis of B. thetaiotaomicron OMVs, Elhenawy et al. reported the association of 60 proteins with OMVs that were not detectable in the bacterial outer membrane, indicating specific targeting to OMVs (74). Of these proteins, two were selected: the hypothetical protein BT1491 and the SusD (starch binding protein) homolog BT3238. Both were detected not only by sensitive MS approaches but also as strong bands on a Coomassie gel of purified OMVs. This indicates high concentrations rather than contaminations due to poor purification. Further, the inulinase BACOVA_04502, a B. ovatus OMV protein (141), was chosen and found to be substantially enriched in OMVs after expression in B. fragilis, suggesting its universal sorting mechanism among Bacteroides spp. (74). BT1491, BT3238, and BACOVA_04502 are in the following referred to as OMV proteins. To examine whether these proteins are able to guide heterologous proteins into OMVs, each was genetically fused to the N-terminus of the luciferase reporter NanoLuc (NL, 19 kDa) with a C-terminal hexahistidine tag (His tag, H₆) (SEQ ID NO: 49). As negative control, a His-tagged NL without the OMV protein was used. Previous studies in other bacteria have shown that fusion proteins with N-terminal OMV-proteins are exported more reliably than with C-terminal (83, 84).

The fusion proteins and the negative control were expressed in B. thetaiotaomicron, and the presence of NL in cell lysates as well as OMV-containing culture supernatants was determined. As depicted in FIG. 3, the activity of NL in culture supernatants normalized by OD600 increased by 6-, 10-, or 125-fold when fused to BT1491, BT3238, or BACOVA_04502, respectively. This indicates that the OMV proteins foster NL secretion. In contrast, NL activity in cell lysates was not substantially affected.

Bioinformatics-Based Approach to Identify Signal Peptides in B. thetaiotaomicron OMV Proteins.

Next, the OMV proteins were evolved towards efficient secretion with preferably small fusion tags. The relatively large proteins BT1491, BT3238, and BACOVA_04502 (256, 519, and 650 amino acids, respectively) elevate the risk of misfolding or steric hindrance of their fusion partners. This may impede their function or have a negative impact on secretion efficiency. Further, the intrinsic function of BT1491 is not elucidated and might have unexpected side effects or harm host cells in future applications.

It was reasoned that the N-terminal regions of the proteins are crucial for their secretion in OMVs, since certain N-terminal amino acid patterns, so called signal peptides, are essential and sufficient to direct proteins to the general secretory pathway, the periplasm, or membrane (142). To test this hypothesis, the amino acid sequences of the OMV proteins were analyzed for characteristic features and conserved sequence patterns using bioinformatics tools. Although no universal consensus sequence exists in signal peptides of Gram-negative bacteria, they typically display regions of distinct polarities or charges and a certain degree of conservation at the −3 and +1 positions relative to the cleavage site separating signal peptide and mature protein (143, 144). Further, secreted proteins cleaved by signal peptidase (SP)I behind an alanine are distinguished from lipoproteins cleaved by SPII before the cysteine that anchors the mature protein into the membrane by its attached lipid moiety.

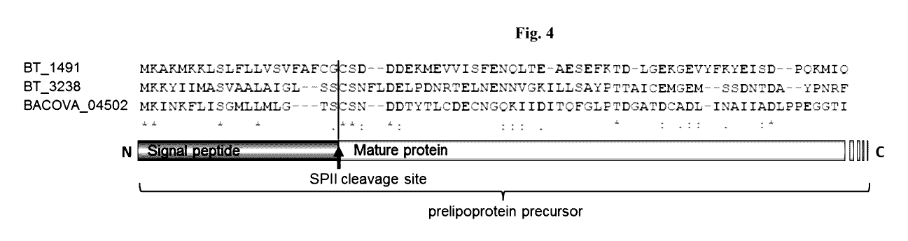

Four bioinformatics software (LipoP 1.0 (144), SignalP 4.1 (145), Phobius (146), and SignalBLAST (147)) predicted N-terminal signal peptides in the three OMV proteins characteristic for proteins of the secretory pathway. Further, they were predicted to be lipoproteins with SPII specific cleavage sites (before a conserved cysteine) by the LipoP tool, which discriminates between signal peptides of secreted proteins and lipoproteins as well as N-terminal transmembrane helices in Gram-negative bacteria (144) (FIG. 4).

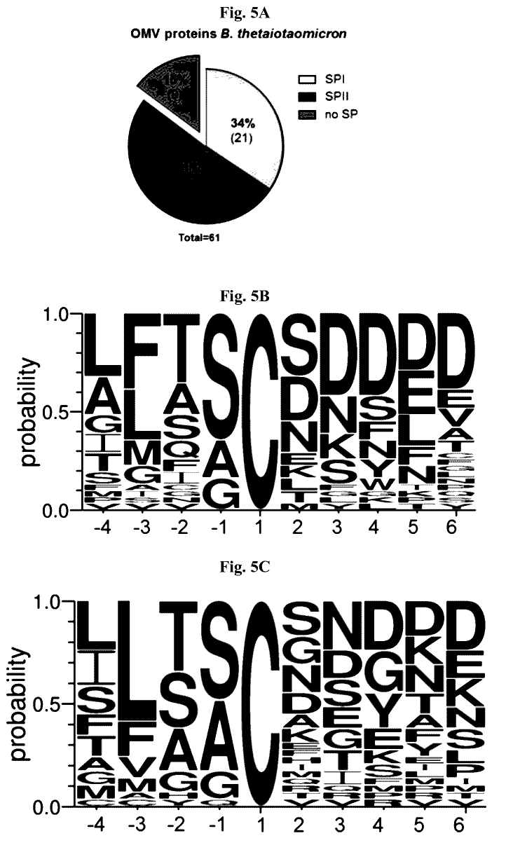

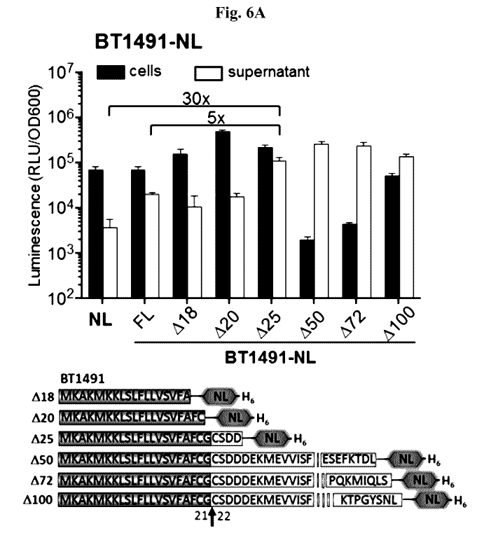

To investigate whether lipoproteins are enriched in Bacteroides OMVs and whether a consensus sequence specific for OMV lipoproteins exists in their signal peptides, all B. thetaiotaomicron OMV proteins were compared with proteins exclusively detected in the OM in the study of Elhenawy et al. (74) using bioinformatics analysis. The protein BACOVA_04502 from B. ovatus that was used in this study was also included in the list of OMV proteins. Only 4 out of 61 Bacteroides OMV proteins had no predicted signal peptide by neither of the four tools. 31 OMV proteins were predicted lipoproteins by the LipoP software with cleavage sites occurring within the first 17-42 amino acids (aa) (FIG. 5A). Of 10 proteins exclusively detected in the OM and not in OMVs, 3 were lipoproteins. Next, whether an OMV-targeting motif in the OMV lipoprotein exists which differs from signal peptides of other B. thetaiotaomicron lipoproteins was examined. Therefore, the OMV lipoprotein sequences were compared to lipoproteins not found in OMVs as negative controls. The UniProt database (148) was searched for (putative) lipoproteins in B. thetaiotaomicron and 26 lipoproteins for which LipoP predicted SPII-cleavable signal peptides and were not detected in OMVs by Elhenawy et al. (74) were obtained. The 26 lipoproteins found in UniProt and the 3 lipoproteins detected exclusively in the OM by Elhenawy et al. were grouped as ‘other lipoproteins’ as compared to OMV lipoproteins. The characteristic structure of signal peptides with a positively charged N-terminal region, a central hydrophobic H-region, and a polar C-terminal region containing the signal peptidase cleavage site can be seen.

To compare amino acid sequences surrounding the SPII-cleavage site of OMV lipoproteins with those of other lipoproteins, sequence logos that indicate 4 aa before and 6 aa after the cleavage site were generated for both groups (FIGS. 5B-5C). Both sequence logos show an enrichment of hydrophobic amino acids N-terminal to the cleavage site; e.g. position −3 is enriched in leucine and phenylalanine, which are conserved amino acids in lipoprotein signal peptides (144). C-terminal to the cleavage site, the negatively charged amino acids aspartic acid and glutamic acid are enriched. It is intriguing that both groups—but OMV lipoproteins to a higher extent—reveal an aspartic acid at position +2 in some of their mature proteins. At this position, aspartic acid is suggested to direct lipoproteins to the inner rather than the outer membrane in other Gram-negative bacteria (149). While, the ‘other lipoprotein’ group may contain lipoproteins of the inner membrane, OMVs do not have an inner membrane.

In conclusion, the sequence logos do not clearly point towards the existence of a conserved amino acids sequence adjacent to the cleavage site that targets particular lipoproteins to OMVs. However, a relative enrichment in aspartic acid was seen in the N-terminus of mature lipoproteins targeted to OMVs.

Truncated OMV Proteins Target NanoLuc to OMV-Containing Supernatants.

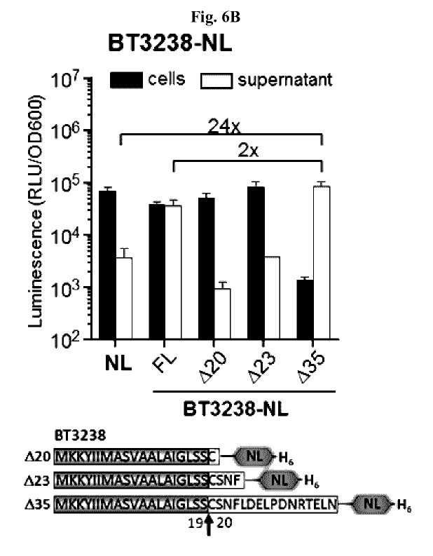

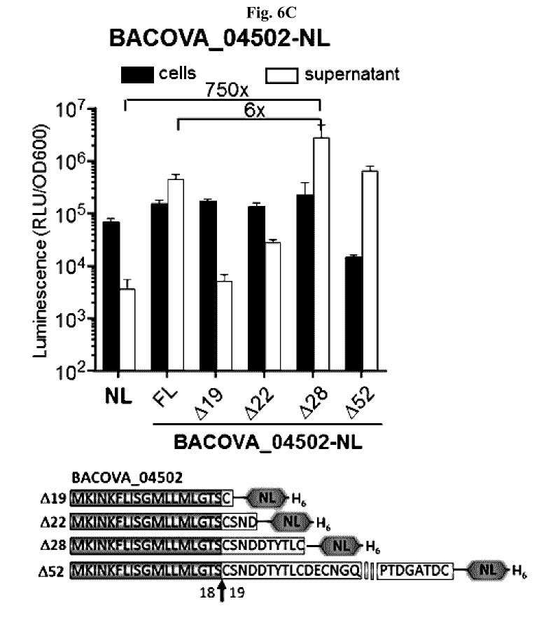

Based on the signal peptides predicted by bioinformatics tools, a short N-terminal sequence was experimentally tested to determine if it is sufficient for protein export into OMVs or if interactions mediated by the whole protein are required. To optimize the tags for small size and export efficiency, truncations of different length between 18 and 100 N-terminal amino acids were created (schemes in FIGS. 6A-6C). The truncations affected NL luminescence in cell lysates and supernatants (FIGS. 6A-6C). In all three tested proteins, the smallest peptide tags (<23 amino acids) decreased luminescence in the supernatant compared to their respective full length (FL) equivalent. With increasing lengths, the luminescence rose to a maximum at 28-50 aa. Each tag had a minimal length necessary to have at least the same secretion proficiency as its full length equivalent indicating a signal peptide activity of the N-termini.

For BT1491, this minimal length was 25 amino acids (BT1491Δ25); for BT3238 35 aa (BT3238Δ35) and for BACOVA_04052 28 aa (BACOVA_04052Δ28). BT1491Δ25, BT3238Δ35, and BACOVA_04502Δ28, hereinafter referred to as OMV tags, were chosen for the following experiments, as they feature a good balance between small size and export efficiency. Importantly, the tags showed higher export efficiencies than the FL proteins, with 5-, 2-, and 6-fold NL activity, respectively, suggesting that the rest of the protein does not mediate OMV translocation. Notably, for efficient export the tags required more amino acids than the predicted signal peptide only. Strikingly, the most efficient tags (BT1491Δ50, BT3238Δ35, BACOVA_04502Δ28) substantially reduced the intracellular luminescence showing that secretion via tags is an effective means of shedding proteins from the cell.

To conclude, heterologous proteins were enriched in OMV-containing supernatants after fusion to either of the three OMV proteins BT1491, BT3238, and BACOVA_04502, as well as optimized C-terminally truncated versions with increased export efficiency over the FL proteins. Further, a minimal sequence tag that is essential for secretion exists for each protein, indicating the existence of an N-terminal OMV signal peptide with little or no role for the rest of the protein.

Functional Heterologous Proteins Fused to OMV Tags Translocate to OMVs.

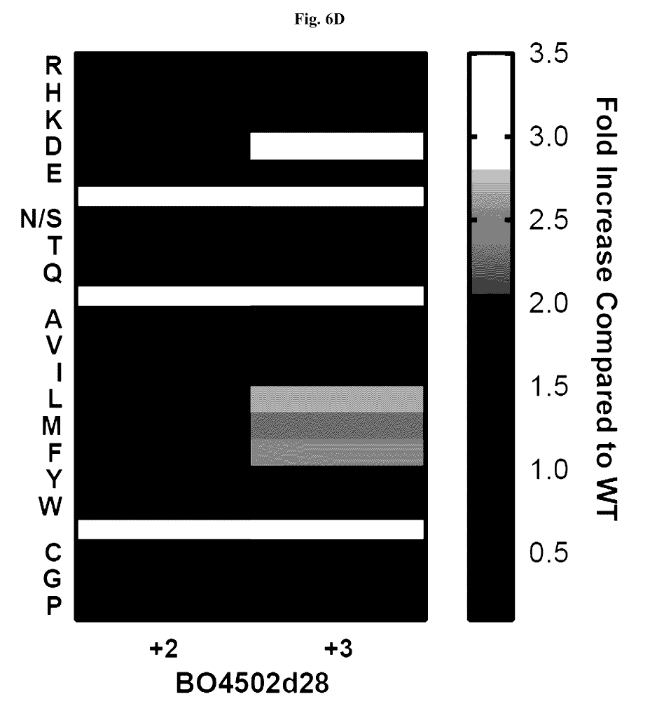

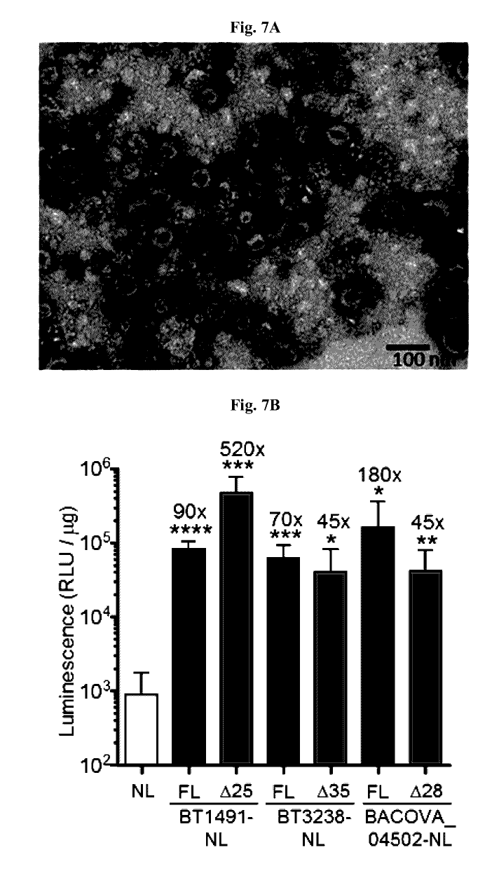

The previous experiments showed that the protein of interest was secreted in supernatants. However, the enrichment in culture supernatants does not distinguish between OMV- and soluble, non-OMV-mediated secretion. Hence, purified OMVs harvested from cell-free supernatants by high-speed centrifugation were analyzed next. The successful enrichment of OMVs in the pellet was confirmed by electron microscopy (FIG. 7A). Luciferase activity in OMVs with NL fused to an OMV protein or its truncation was significantly increased in OMVs with untagged NL (FIG. 7B). Further, fusion proteins were analyzed with Western blot analysis (FIG. 7C). The size of the detected proteins corresponded to the size of the OMV tag-NL-His₆ (SEQ ID NO: 13) fusion proteins as calculated at/web.expasy.org/compute_pi/.

In conclusion, these results indicate that the NL reporter is secreted via OMVs by fusing it to B. thetaiotaomicron FL OMV proteins or optimized OMV tags without disturbing its luciferase functionality. These experiments show that the 25-35 N-terminal amino acids are sufficient for targeting to OMVs.

OMV Tags are Universal Across Bacteroides Spp.

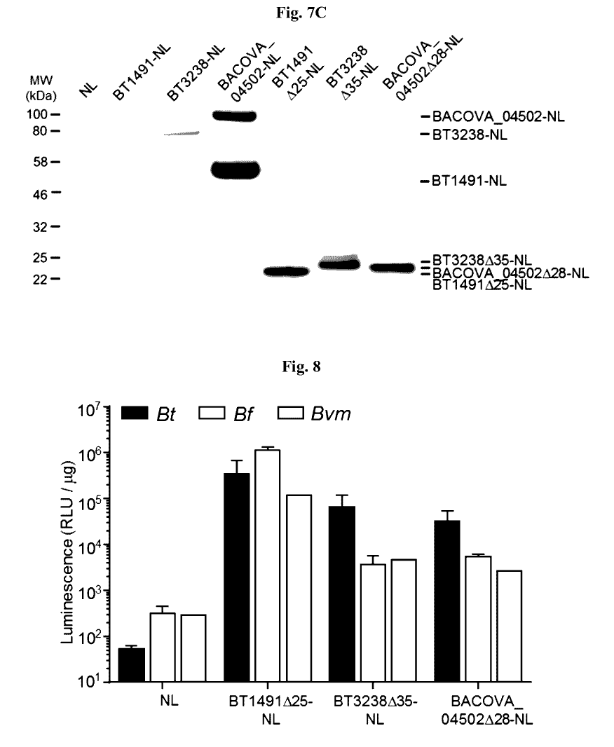

OMV tag-NL fusions were expressed in B. fragilis and B. vulgatus to test whether the tags mediate export to OMVs in other Bacteroides species than B. thetaiotaomicron. While the B. thetaiotaomicron and B. fragilis strains are both purchased human isolates, B. vulgatus was isolated in-house from feces of Swiss Webstar mice. Purified OMVs of B. fragilis and B. vulgatus showed increased luminescence when expressing fusion proteins of OMV tags with NL compared to NL alone (FIG. 8). This indicates that the export via OMV tags, especially BT1491Δ25, is compatible with B. fragilis and murine B. vulgatus. It enables to choose between different Bacteroides species and use the one most sufficient in recombinant protein production or most tolerable in vivo.

### Example 2: Localization of OMV Cargo Packaged by OMV Tags

Since the three OMV tags are predicted to harbor signal peptides characteristic for lipoproteins, it is likely that they are anchored to the OMV membrane. The orientations of the OMV tag-NL fusion proteins in the membrane were investigated next. Although lipoproteins in Gram-negative bacteria have been predominantly found anchored in the inner leaflet of the outer membrane (150), a high percentage of Bacteroides lipoproteins have been found cell surface-exposed (151). BT3238 is a homolog of SusD, a starch-binding protein, and is presumably surface-exposed. Surface-exposed fusion proteins would facilitate interaction of protein therapeutics with cell surface receptors.

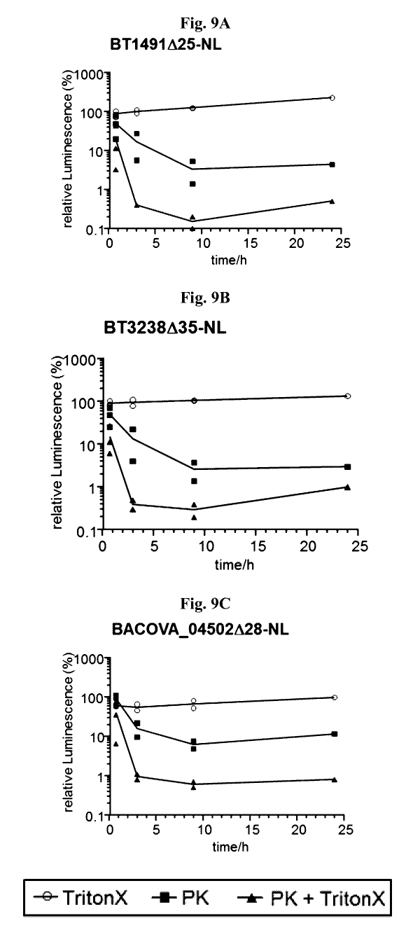

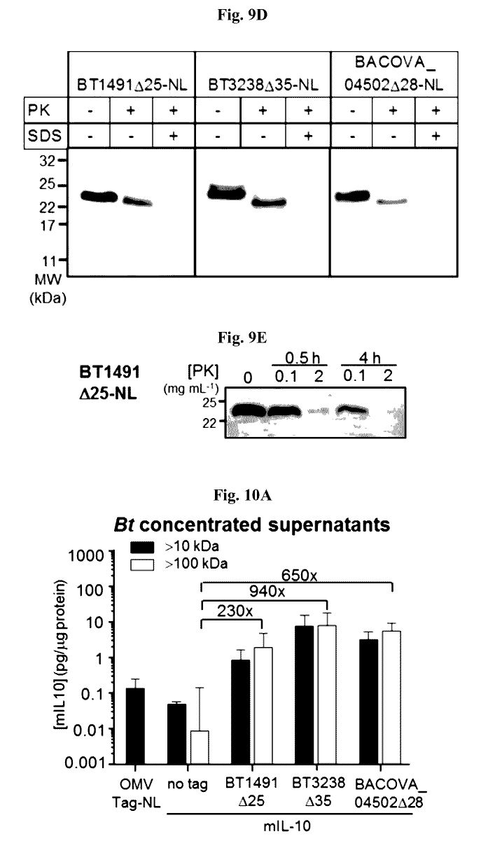

Proteinase K protection assays were performed on OMVs purified from B. thetaiotaomicron cultures expressing the three OMV tag-NL fusion proteins to examine whether they are accessible on the OMV surface or protected by the vesicle structure (FIG. 9A). Treating OMVs with proteinase K decreased the NL luminescence in all three samples compared to untreated OMVs over a course of 24 hours (FIGS. 9A-9C). Within 3 hours incubation, only 13-17% of NL activity remained for all tags. After 9 hours the NL activity stabilized to 3-6% of the untreated activity. Disruption of the membrane integrity by Triton-X, however, abolished luminescence to percentages below 1 after 3 hours. This indicates that NL was not fully degraded by proteinase K when no membrane-disrupting detergent was applied. Luminescence was not decreased by Triton-X alone. Slight increases in relative luminescence between 9 and 24 hours might be due to evaporation of liquid in the reaction solution leading to higher sample concentration. However, it remains to be determined, if the vesicle structure was still impermeable and the outer membrane was not destroyed by degradation of surface-exposed proteins by proteinase K. Control experiments with proteins that are known to be OMV surface exposed and contained within the OMV, respectively, are necessary.

Consistent with the luminescence data, Western blots revealed lower protein amounts after incubation with proteinase K for 30 min compared to without treatment (FIG. 9D). Increase of proteinase K concentration or extension in time augmented the digestion of BT1491Δ25-NL (FIG. 9E) and BACOVA_04502Δ28-NL, so that it was barely detectable after 4 h incubation with 2 mg mL⁻¹ PK. Interestingly, bands of fusion proteins decreased 1-2 kDa in size following treatment with proteinase K, particularly for BT3238Δ35-NL and BACOVA_04502Δ28-NL.

Fusion tags might be displayed at the cell surface and the OMV tags mediate anchorage in the outer leaflet of the OMV membrane. However, fusion proteins are substantially more stable when OMVs were intact. Up to 10% remained even after 24 hours PK digestion pointing towards a protective function of OMVs. Here, it is worth noting, that B. thetaiotaomicron produces a thick capsule that covers not only its cell envelope but also its OMVs (152) and might limit access of proteinase K to surface proteins leading to incomplete digestion. Alternatively, a proportion of OMV tags could have failed to display the fused NL on the surface and rather remain inside the lumen like shown for an E. coli fusion proteins in a previous study (84).

### Example 3: IL-10 Secretion in OMVs

Having established an OMV-based secretion system for NL in the gut commensal B. thetaiotaomicron, it was utilized for the secretion of a therapeutic protein to treat intestinal disorders. Reduced levels of the immunoregulatory cytokine interleukin (IL)-10 have been linked to several intestinal diseases like inflammatory bowel diseases (IBD) (124, 125, 127, 153, 154). Therefore, the murine IL-10 (mIL-10) cytokine were genetically fused to the three OMV tags, and these fusion proteins were expressed in B. thetaiotaomicron and B. vulgatus. OMV tag-NL fusions and untagged mIL-10 which is not directed to OMVs were used as negative controls.

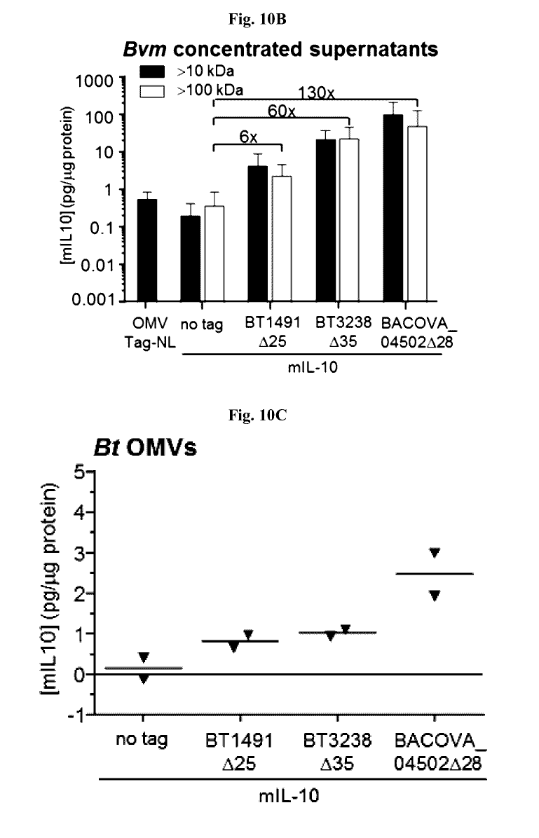

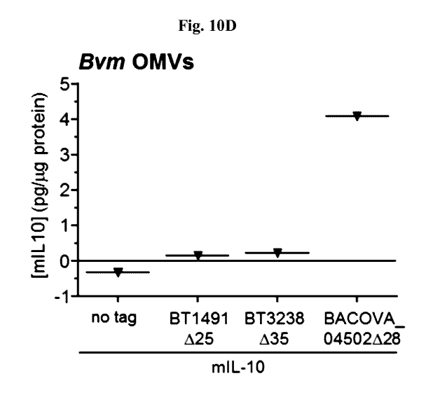

B. thetaiotaomicron cultures were grown for 6 h and B. vulgatus cultures for 20 h to obtain comparable OD600. Culture supernatants were concentrated using centrifugal filter tubes with either 10 or 100 kDa cut-off membranes, which retain large protein complexes and OMVs. Soluble dimeric IL-10 (37 kDa) is retained by the 10 kDa cut-off membrane, whereas it passes the 100 kDa membrane, and is therefore found in the >10 kDa but not in the >100 kDa fraction. Concentrates were analyzed for mIL-10 concentration [mIL-10] by sandwich enzyme-linked immunosorbent assay (ELISA) and normalized to total protein concentrations as determined by bradford assays (FIGS. 10A-10B). Fusion to OMV tags enriches mIL-10 in both, B. thetaiotaomicron and B. vulgatus supernatants, as compared to untagged mIL10: In >100 kDa fractions [mIL-10] was increased up to 940-fold for BT3238Δ35-mIL10 in B. thetaiotaomicron and up to 130-fold for BACOVA_04502-mIL10 in B. vulgatus. [mIL-10] was not substantially increased in >10 kDa compared to >100 kDa fractions either in B. thetaiotaomicron or in B. vulgatus, indicating that mIL-10 was not solubly secreted in large amounts but mostly in association with OMVs. Highest mIL-10 concentration was 100 pg/μg for BACOVA_04502Δ28 in >10 k fractions of B. vulgatus culture supernatant, which corresponds to ca. 50 ng mIL-10 in 1 ml unconcentrated culture supernatant. Together these results indicate that mIL-10 is secreted only when fused to OMV tags and does barely leak from cells when untagged.

To determine if the mIL-10 found in concentrated culture supernatants is associated with OMVs and is not a result of mIL-10 aggregates heavier than 100 kDa, OMVs were purified by high-speed centrifugation. [mIL-10] in purified OMVs were lower than in concentrated supernatants but still detectable (FIGS. 10C-10D). In B. thetaiotaomicron OMVs the mIL-10 concentration (2.5 pg/μg) was highest when expressing BACOVA_04502Δ28-mIL10 fusions. The same fusion protein was present in B. vulgatus OMVs with a [mIL-10] of 4 pg/μg. This corresponds to about 0.3 ng OMV-associated mIL-10 in 1 mL unconcentrated culture supernatant. These results indicate that mIL-10 can be targeted to B. thetaiotaomicron and B. vulgatus OMVs.

### Example 4: In Vivo Colonization and Tolerance of Engineered Bacteroides Species in Mice with DSS-Induced Experimental Colitis

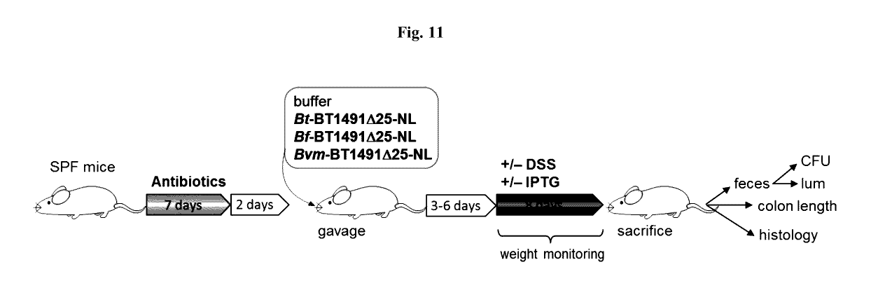

Next, whether it will be conceivable to deploy Bacteroides spp. in vivo in future applications was assessed. Therefore, whether engineered Bacteroides species are able to stably colonize the intestine and whether they are tolerated by mice were investigated. As Bacteroides spp. are unable to colonize the gut of specific-pathogen-free (SPF) mice per se (28), colonization was promoted by antibiotic treatment for 7 days without sterilizing the intestine (155). Subsequently, mice were orally gavaged with B. thetaiotaomicron, B. fragilis, or B. vulgatus mouse isolate expressing the BT1491Δ25-NL fusion protein (Bt-BT1491Δ25-NL, Bf-BT1491Δ25-NL, Bvm-BT1491Δ25-NL). 4-7 days after bacterial gavage, experimental colitis was induced by administration of dextran sulfate sodium (DSS) in drinking water for 8 days. Simultaneously, IPTG was administered to induce the expression of the BT1491Δ25-NL fusion protein (FIG. 11).

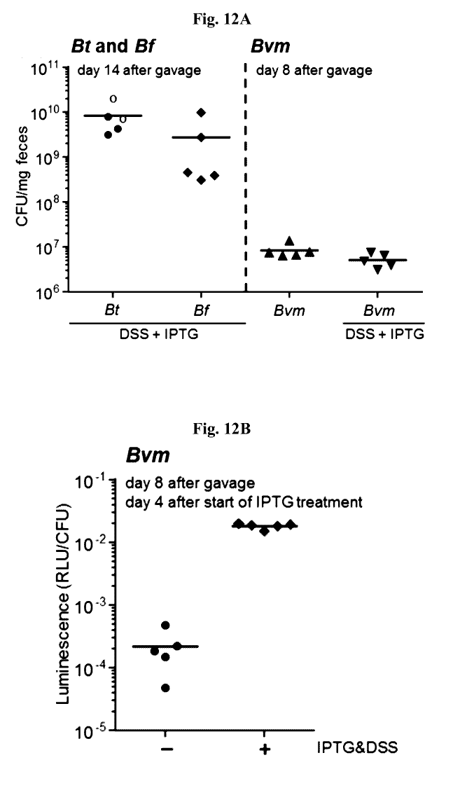

First, it was shown that mice were successfully colonized with Bacteroides spp. and that DSS and IPTG application did not interfere with colonization. Engineered Bacteroides were identified by plating mice feces on selective media (BHIS+Gm+Em) for Bacteroides and plasmid selection. B. vulgatus was still detected in feces 8 days after gavage, and B. thetaiotaomicron and B. fragilis 14 days after gavage (FIG. 12A). In contrast, feces of mice gavaged with sucrose buffer instead of bacteria did not reveal Bacteroides colonies (data not shown). Further, luminescence was detected in feces 4 days after the start of IPTG administration, indicating that the expression of BT1491Δ25-NL fusion protein was successfully induced in vivo (FIG. 12B).

To assess how well Bacteroides spp. are tolerated under inflammatory conditions in mice, the impact of Bacteroides colonization on intestinal inflammation in the DSS model of colitis was investigated. DSS-induced colitis is a widely used mouse model for experimental intestinal colitis which is thought to be induced by direct toxicity of DSS to colonic epithelial cells of the basal crypts (156, 157). Colitis severity was evaluated by the macroscopic feature of a reduced colon length and histological features of the colon such as inflammatory infiltrates, edema, and epithelial defects scored in a blinded fashion. Further, wellbeing of mice was monitored by recording the mouse weight over the experimental course.

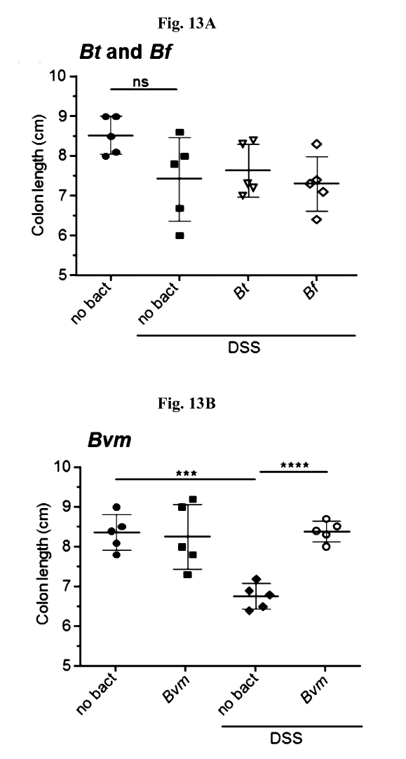

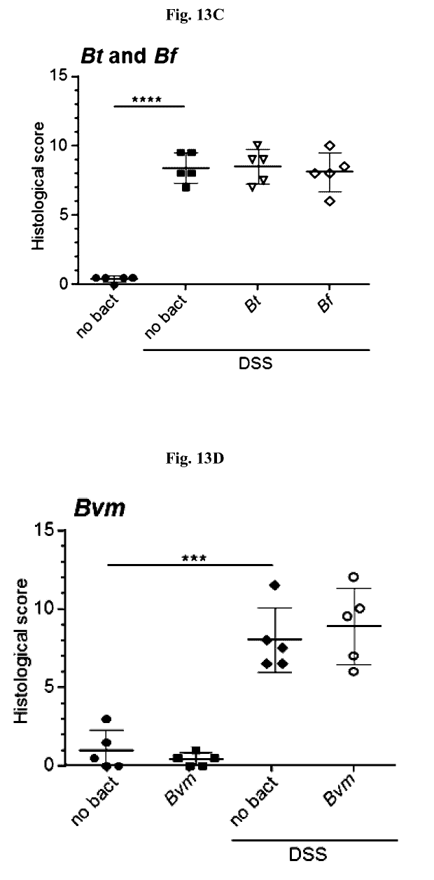

Colitis was successfully induced, as non-colonized mice that where administered 3% DSS for 7 day in their drinking water revealed shortened colons (FIGS. 13A-13B) and significantly more severe histological scores (FIGS. 13C-13D) compared to untreated controls. Intestinal colonization with Bacteroides spp. did not exacerbate inflammation. No significant difference in colon lengths (FIGS. 13A-13B) and no increased histological scores (FIGS. 13C-13D) compared to uncolonized control were detected. Colon length of mice colonized with B. vulgatus was even increased compared to uncolonized control. Additionally, Bacteroides spp. had no adverse effect on overall mouse health as determined by body weight. Colonization with Bacteroides spp. did not substantially reduce body weight compared to uncolonized controls.

Mice were stably colonized with Bacteroides spp. over the course of the experiment and expression of recombinant proteins was successfully induced. Bacteroides colonization was well tolerated and had no detrimental effect on intestinal inflammation in a DSS-induced colitis model.

The potential to deploy engineered microbes as intestinal drug delivery systems in human medicine brought forth an important emerging field of research. It enables a targeted, low-dose, and cost-effective treatment by using orally administered microbes that produce and deliver therapeutic agents directly to the site of action. The majority of work has been done in E. coli (131, 135, 136) and L. lactis (127, 132), which are not capable to continuously deliver drugs in high therapeutic doses due to their low abundance in the GI tract and their inability to colonize the gut, respectively. In this work, the basis for the development of a novel therapeutic protein delivery system to treat intestinal disorders was established. The aim was to enable the use of outer membrane vesicles (OMVs) produced by gut commensals of the Bacteroides species as delivery vehicles for therapeutic proteins to the intestine. Therefore, Bacteroides spp. that naturally reside in the intestinal mucus layer were engineered to generate OMVs that carry heterologous proteins which can be delivered to host cells in vivo.

Three OMV proteins—BT1491, BT3238, and BACOVA_04502—found in natural Bacteroides OMVs that translocated the reporter NanoLuc (NL) to OMVs when genetically fused to its N-terminus were identified. These proteins were further optimized by truncation to small fusion tags of 25-35 amino acids (OMV tags) while maintaining the ability to target NL for OMV-based secretion in B. thetaiotaomicron, B. fragilis, and a murine isolate of B. vulgatus. The three OMV tags were N-terminal regions of OMV lipoproteins comprising a predicted signal peptide and 4-16 additional amino acids. Our data suggest that the cargo is carried on the OMV surface via a lipid anchor in the outer leaflet of the OMV membrane. In a proteinase K assay, the speed of NL degradation was decreased by intact OMVs and 3-6% of the cargo remained 24 h after proteinase K addition. Additionally, translocation of the therapeutic protein IL-10 to OMVs in concentrations up to 0.3 ng OMV-associated IL-10 per mL culture supernatant in a proof-of-concept experiment were achieved. These results indicate that it is possible to target heterologous proteins to Bacteroides OMVs. For future in vivo applications, all three tested Bacteroides spp.—B. thetaiotaomicron, B. fragilis, and B. vulgatus mouse isolate—were identified as suitable chassis that stably colonized the gut and had no detrimental effects on mice in an experimental colitis model. This will be the basis for further in vivo work to examine therapeutic effects of OMV-associated proteins.

### Materials

TABLE 1
Plasmids

| Identifier | Plasmid | Description |
|---|---|---|
| jfrP1 | pNBU1-L23R-NL-h | IPTG-inducible NanoLuc expression, pNBU1 |
| | | backbone, β-Lactamase and ErmG resistance genes |
| jfrP2 | pNBU1-L23R-BT1491-h | IPTG-inducible BT1491 expression, pNBU1 |
| | | backbone, β-Lactamase and ErmG resistance genes |
| jfrP3 | pNBU1-L23R-BT1491NL-h | IPTG-inducible BT1491-NL expression, pNBU1 |
| | | backbone, β-Lactamase and ErmG resistance genes |
| jfrP4 | pNBU1-L23R-BT3238-h | IPTG-inducible BT3238 expression, pNBU1 |
| | | backbone, β-Lactamase and ErmG resistance genes |
| jfrP5 | pNBU1-L23R-BT3238-NL-h | IPTG-inducible BT3238-NL expression, pNBU1 |
| | | backbone, β-Lactamase and ErmG resistance genes |
| jfrP6 | pNBU1-L23R-BT1491-del18-NL-h | IPTG-inducible BT1491Δ18-NL expression, pNBU1 |
| | | backbone, β-Lactamase and ErmG resistance genes |
| jfrP7 | pNBU1-L23R-BT1491-del25-NL-h | IPTG-inducible BT1491Δ25-NL expression, pNBU1 |
| | | backbone, β-Lactamase and ErmG resistance genes |
| jfrP8 | pNBU1-L23R-BT1491-del50-NL-h | IPTG-inducible BT1491Δ50-NL expression, pNBU1 |
| | | backbone, β-Lactamase and ErmG resistance genes |
| jfrP9 | pNBU1-L23R-BT1491-del72-NL-h | IPTG-inducible BT1491Δ72-NL expression, pNBU1 |
| | | backbone, β-Lactamase and ErmG resistance genes |
| jfrP10 | pNBU1-L23R-BT1491-del100-NL-h | IPTG-inducible BT1491Δ100-NL expression, pNBU1 |
| | | backbone, β-Lactamase and ErmG resistance genes |
| jfrP11 | pNBU1-L23R-BT1491-del20-NL-h | IPTG-inducible BT1491Δ20-NL expression, pNBU1 |
| | | backbone, β-Lactamase and ErmG resistance genes |
| jfrP12 | pNBU1-L23R-BT3238-del20-NL-h | IPTG-inducible BT3238Δ20-NL expression, pNBU1 |
| | | backbone, β-Lactamase and ErmG resistance genes |
| jfrP13 | pNBU1-L23R-BT3238-del23-NL-h | IPTG-inducible BT3238Δ23-NL expression, pNBU1 |
| | | backbone, β-Lactamase and ErmG resistance genes |
| jfrP14 | pNBU1-L23R-BT3238-del35-NL-h | IPTG-inducible BT3238Δ35-NL expression, pNBU1 |
| | | backbone, β-Lactamase and ErmG resistance genes |
| jfrP15 | pNBU1-L23R-BO04502-del19-NL-h | IPTG-inducible BACOVA_04502Δ19-NL expression, |
| | | pNBU1 backbone, β-Lactamase and ErmG resistance |
| | | genes |
| jfrP16 | pNBU1-L23R-BO04502-del22-NL-h | IPTG-inducible BACOVA_04502Δ22-NL expression, |
| | | pNBU1 backbone, β-Lactamase and ErmG resistance |
| | | genes |
| jfrP17 | pNBU1-L23R-BO04502-del28-NL-h | IPTG-inducible BACOVA_04502Δ28-NL expression, |
| | | pNBU1 backbone, β-Lactamase and ErmG resistance |
| | | genes |
| jfrP18 | pNBU1-L23R-BO04502-del52-NL-h | IPTG-inducible BACOVA_04502Δ52-NL expression, |
| | | pNBU1 backbone, β-Lactamase and ErmG resistance |
| | | genes |
| jfrP20 | pNBU1-L23R-BT1491-del25-IL10 | IPTG-inducible BT1491Δ25-IL-10 expression, |
| | | pNBU1 backbone, β-Lactamase and ErmG resistance |
| | | genes |
| jfrP23 | pNBU1-L23R-BT3238-del35-IL10 | IPTG-inducible BT3238Δ35-IL-10 expression, |
| | | pNBU1 backbone, β-Lactamase and ErmG resistance |
| | | genes |
| jfrP26 | pNBU1-L23R-BO04502-del28-IL10 | IPTG-inducible BACOVA_04502Δ28-IL-10 |
| | | expression, pNBU1 backbone, β-Lactamase and |
| | | ErmG resistance genes |
| jfrP41 | pNBU1-L23R-IL10 | IPTG-inducible IL-10 expression, pNBU1 backbone, |
| | | β-Lactamase and ErmG resistance genes |

TABLE 2
Primers for Cloning (Integrated DNA Technologies, Coralville, Iowa, USA)

| | | | | SEQ ID |
|---|---|---|---|---|
| Identifier | Target | Sequence | Direction | NO: |
| jfrD1 | BT1491delta18aa | CCACCGCCACCGCTACCGCCACCGCCAGCGAA | rv | 17 |
| | | TACTGATACCAGTAAGAATAAAGATAAC | | |
| jfrD2 | BT1491delta25aa | TGCCACCGCCACCGCTACCGCCACCGCCATCA | rv | 18 |
| | | TCACTACATCCACAAAAAGCGAATACTG | | |
| jfrD3 | BT1491delta50aa | TGCCACCGCCACCGCTACCGCCACCGCCTAAA | rv | 19 |
| | | TCTGTTTTAAATTCACTCTCTGCTTCAG | | |
| jfrD4 | BT1491delta72aa | ATACTGCCACCGCCACCGCTACCGCCACCGCC | rv | 20 |
| | | AGATAGCTGTATCATCTTTTGCGGATCG | | |
| jfrD5 | BT1491delta100aa | CATACTGCCACCGCCACCGCTACCGCCACCGC | rv | 21 |
| | | CGAGGTTGCTATAGCCGGGAGTCTTTAC | | |
| jfrD6 | BT1491deltax | GGCGGTGGCGGTAGCGGTGG | fw | 22 |
| jfrD7 | BT3238bb-h | CGGATTAAAACCAAACCCTAGAACTAAAGGTT | fw | 23 |
| | | CTGGTCATCATCACCATCACCACTAATG | | |
| jfrD8 | BT3238bb-NL-h | TGTTATTTCAGCCGGATTAAAACCAAACCCTA | fw | 24 |
| | | GAACTAAAGGCGGTGGCGGTAGCGGTGG | | |
| jfrD9 | BT3238bb | TGGCCATTATTATGTATTTCTTCATGTTTATATT | rv | 25 |
| | | ATTTATATTTGTTTGACGAGAATATC | | |
| jfrD10 | BT3238ins | CTCGTCAAACAAATATAAATAATATAAACAAG | fw | 26 |
| | | AAATACATAATAATGGCCAGTGTGGCCG | | |
| jfrD11 | BT3238ins-h | AGTGGTGATGGTGATGATGACCAGAACCTTTA | rv | 27 |
| | | GTTCTAGGGTTTGGTTTTAATCCGGCTG | | |
| jfrD12 | BT3238ins-NL-h | TGCCACCGCCACCGCTACCGCCACCGCCTTTA | rv | 28 |
| | | GTTCTAGGGTTTGGTTTTAATCCGGCTG | | |
| jfrD13 | BT1491delta20aa | CACCGCCACCGCTACCGCCACCGCCACAAAAA | rv | 29 |
| | | GCGAATACTGATACCAGTAAGAATAAAG | | |
| jfrD14 | BT3238delta20aa | AACCATACTGCCACCGCCACCGCTACCGCCAC | rv | 30 |
| | | CGCCACACGATGAGAGCCCTATTGCGAG | | |
| jfrD15 | BT3238delta23aa | ACTGCCACCGCCACCGCTACCGCCACCGCCGA | rv | 31 |
| | | AATTAGAACACGATGAGAGCCCTATTGC | | |
| jfrD16 | BT3238delta35aa | CATACTGCCACCGCCACCGCTACCGCCACCGC | rv | 32 |
| | | CATTCAGCTCGGTACGATTGTCGGGAAG | | |
| jfrD17 | B004502delta19aa | ACCATACTGCCACCGCCACCGCTACCGCCACC | rv | 33 |
| | | GCCGCAAGATGTGCCCAACATCAGGAGC | | |
| jfrD18 | B004502delta22aa | ATACTGCCACCGCCACCGCTACCGCCACCGCC | rv | 34 |
| | | GTCATTGCTGCAAGATGTGCCCAACATC | | |
| jfrD19 | B004502delta28aa | TACTGCCACCGCCACCGCTACCGCCACCGCCA | rv | 35 |
| | | CAGAGAGTATAGGTATCGTCATTGCTGC | | |
| jfrD20 | B004502delta52aa | CATACTGCCACCGCCACCGCTACCGCCACCGC | rv | 36 |
| | | CACAATCAGTGGCCCCATCGGTTGGAAG | | |
| jfrD21 | IL-10 w/o SP | AAGGCGGTGGCGGTAGCGGTGGCGGTGGCAGT | fw | 37 |
| | | ATGTCACGGGGTCAGTACAGCCGCGAAG | | |
| jfrD22 | Linker + BT1491 | TCTTGACGGTATCACCCTCATCGAAAAAGGCG | fw | 38 |
| | | GTGGCGGTAGCGGTGGCGGTGGCAGTAT | | |
| jfrD23 | Linker + BT3238 | CGGATTAAAACCAAACCCTAGAACTAAAGGCG | fw | 39 |
| | | GTGGCGGTAGCGGTGGCGGTGGCAGTAT | | |
| jfrD24 | Linker + BO04502 | TATATTACATTATCTAAGCGCTAAGAAAGGCG | fw | 40 |
| | | GTGGCGGTAGCGGTGGCGGTGGCAGTAT | | |
| jfrD25 | 6*His + IL10 | CTACATGATGATTAAAATGAAAAGCTAAGGTT | fw | 41 |
| | | CTGGTCATCATCACCATCACCACTAATG | | |
| jfrD28 | IL10 + 6*His | TGGTGATGGTGATGATGACCAGAACCGCTTTT | ry | 42 |
| | | CATTTTAATCATCATGTAGGCTTCAATG | | |
| jfrD30 | 6*His-Terminator | GTGCAGTTCATTAGTGGTGATGGTGATG | rv | 43 |
| jfrD31 | Linker + | ATTCGCTTTTTGTGGATGTAGTGATGATGGCGG | fw | 44 |
| | BT1491del25 | TGGCGGTAGCGGTGGCGGTGGCAGTAT | | |
| jfrD32 | Linker(anneal) + | GCTTCCCGACAATCGTACCGAGCTGAATGGCG | fw | 45 |
| | BT3238del35 | GTGGCGGTAGCGGTGGCGGTGGCAGTAT | | |
| | (overhang) | | | |
| jfrD33 | Linker + | CAGCAATGACGATACCTATACTCTCTGTGGCG | fw | 46 |
| | BO04502del28 | GTGGCGGTAGCGGTGGCGGTGGCAGTAT | | |
| jfrD34 | Linker(anneal) + | CTTCTTATTTCTGTCTTGACGGTATCACCCTCA | fw | 47 |
| | BT1491(overhang) | TCGAAAAAGGCGGTGGCGGTAGCGGTG | | |
| jfrD35 | Linker(anneal) + | ATGTTATTTCAGCCGGATTAAAACCAAACCCT | fw | 48 |
| | BT3238(overhang) | AGAACTAAAGGCGGTGGCGGTAGCGGTG | | |
| jfrD36 | Linker(anneal) + | TCATGACGAAGAATATATTACATTATCTAAGC | fw | 50 |
| | BO04502(overhang) | GCTAAGAAAGGCGGTGGCGGTAGCGGTG | | |
| jfrD37 | Backbone + OMV- | GTTTATATTATTTATATTTGTTTGACGAGAATA | rv | 51 |
| | Tags | TC | | |
| jfrD38 | IL10 w/o Tag | AAAGATATTCTCGTCAAACAAATATAAATAAT | fw | 52 |
| | | ATAAACATGTCACGGGGTCAGTACAGCC | | |

TABLE 3
Reagents

| Reagent | Manufacturer | Catalog number |
|---|---|---|
| Agar | Sunrise Science Products | 1910 |
| Brain Heart Infusion (BHI) | BD Difco | 241830 |
| β-mercaptoethanol | ThermoFisher Scientific, | 21-985-023 |
| | Gibco | |
| BugBuster Protein Extraction Reagent | Novagen, EMD Millipore | 70584 |
| Carbenicillin | VWR | 100219-046 |
| Ciprofloxacin HCl | VWR | AAJ61970-06 |
| Clarity ™ Western ECL Substrate | Bio-Rad | 170-5060 |
| CutSmart ® Buffer | New England BioLabs | B7204S |
| Dextran sulfate sodium (DSS) | Affymetrix | 9011-18-1 |
| DNA Gel Loading Dye (6X) | ThermoFisher Scientific | R0611 |
| DNA Ladder, 2-Log | New England BioLabs | N3200 |
| DpnI | New England BioLabs | R0176S |
| Erythromycin | Sigma Aldrich | E5389 |
| Gentamicin | Sigma Aldrich | G1272 |
| Hemin | Sigma Aldrich | 16009-13-5 |
| Isopropyl β-D-1-thiogalactopyranoside (IPTG) | Sigma Aldrich | I6758 |
| LB | BD Difco | DF0446-07-5 |
| rLysozyme | Novagen, EMD Millipore | 71110 |
| Metronidazole | VWR | 700008-662 |
| NuPAGE ®, LDS Sample Buffer (4X) | Novex, Life Technologies | NP0007 |
| NuPAGE ®, MES SDS Running Buffer (20x) | Novex, Life Technologies | NP0002 |
| NuPAGE ®, 4-12% Bis-Tris Gel | Invitrogen, Thermo Fisher | NP0335 |
| | Scientific | |
| Phenylmethylsulfonyl fluoride (PMSF) | Amresco, Ohio, USA | 329-98-6 |
| Proteinase inhibitor cocktail tablets, EDTA-free | Roche Diagnostics | 11873580001 |
| Proteinase K | Qiagen ® | 19131 |
| Protein standard, broad range, color | New England BioLabs | P7712S |
| SYBR ® Safe DNA Gel Stain | ThermoFisher Scientific | S33102 |
| T5 exonuclease | New England Biolabs | M0363L |
| TAE buffer, 50x | Amresco | K915 |
| Taq ligase | New England BioLabs | |
| Trypticase ™ Peptone | BD BBL ™ | 211921 |
| Yeast extract | BD Bacto | 210929 |

TABLE 4
Kits

| | | Catalog |
|---|---|---|
| Kit | Manufacturer | number |
| Bradford, Quick Start ™, | Bio-Rad | 5000201 |
| protein quantification assay | | |
| ELISA DuoSet, Mouse IL-10 | R&D Systems | DY417-05 |
| ELISA DuoSet, Ancillary Reagent Kit 2 | R&D Systems | DY008 |
| KAPA HiFi PCR kit w/dNTPs | KAPA Biosystems | KK2102 |
| Nano-Glo ® Luciferase Assay | Promega | N1120 |
| Phusion High-fidelityDNA Polymerase | New England | M0530L |
| | Biolabs | |
| QIAprep Spin Miniprep Kit | Qiagen ® | 27104 |
| QIAquick PCR Purification Kit | Qiagen ® | 28104 |
| Taq DNA ligase | New England | M0208L |
| | Biolabs | |
| Vitamin K3, Menadione | Sigma-Aldrich | M5625 |

### Recipes

Gibson assembly master mix: 320 μL 5× Isothermal Master Mix (25% PEG-8000, 500 mM Tris-HCl pH 7.5, 50 mM MgCl2, 50 mM DTT, 5 mM NAD, 1 mM each of the four dNTPs), 0.64 μL 10 U/μL T5 exonuclease, 20 μL 2 U/μL Phusion High-fidelity DNA Polymerase, 0.16 μL 40 000 U/μL Taq DNA Ligase, 860 μL ddH2O
Supplemented brain-heart infusion (BHIS) medium: 37 g/L BHI, 5 g/L yeast extract, 10 mg/L hemin, 1 mg/L Vitamin K3, 0.5 g/L cysteine; diluted in ddH₂O
Supplemented trypticase yeast extract glucose (TYG) medium: 10 g/L trypticase, 5 g/L yeast extract, 1 g/L Na₂CO₃, 10 mM glucose, 80 mM potassium phosphate buffer (pH 7.3), 20 mg/L MgSO₄.7H₂O, 400 mg/L NaHCO₃, 80 mg/L NaCl, 0.0008% CaCl₂, 4 μg/mL FeSO4.7H₂O, 10 mg/L hemin, 1 mg/L Vitamin K3, 0.5 g/L cysteine; diluted in ddH₂O

TABLE 5
Antibodies

| Antibody | Host | Conjugate | Manufacturer | Catalog number |
|---|---|---|---|---|
| anti-6x His tag | mouse | | Abcam | ab18184 |
| anti-Mouse | rabbit | HRP | Abcam | ab6728 |

TABLE 6
Consumables

| Consumable | Manufacturer | Catalog number |
|---|---|---|
| Centrifugal Filter Devices, | EMD Millipore | UFC501096 |
| Amicon Ultra-0.5, Ultracel-10 | | |
| membrane | | |
| Centrifugal Filter Devices, | EMD Millipore | UFC510096 |
| Amicon Ultra-0.5, Ultracel- | | |
| 100 membrane | | |
| Electroporation Cuvette, | Bio-Rad | 15442999 |
| Gene Pulser, 0.2 cm | | |
| iBlot ™ Gel Transfer | ThermoFisher | IB301001 |
| Stacks, nitrocellulose | Scientific | |
| NuPAGE, 4-12% Bis-Tris Gel | Invitrogen, | NP0335 |
| | Thermo Fisher | |
| | Scientific | |
| Stericup ® and Steritop ™ | EMD Millipore | SCHVU01RE |
| Vacuum Driven Sterile Filters, | | |
| 0.45 μm HV Durapore membrane | | |

TABLE 7
Bacterial Strains

| Strain | Provided by |
|---|---|
| Bacteroides fragilis NCTC 9343, | ATCC |
| appendix abscess isolate | |
| Bacteroides thetaiotaomicron | ATCC |
| VPI-5482, human feces isolate | |
| Bacteroides vulgatus, | In-house isolate from |
| mouse feces isolate | feces of female Swiss |
| | Webster mice (Jackson |
| | Laboratory) |
| E. coli S17.1 λpir | gift from group of |
| | Michael Fischbach |

TABLE 8
Mice

| Strain | Sex | Provided by |
|---|---|---|
| Specific-pathogen-free (SPF) | male | Jackson Laboratory |
| C57BL/6 | | |

TABLE 9
Software

| Software | Manufacturer/Reference |
|---|---|
| Geneious ® R8.1.8. | Biomatters Ltd |
| GraphPad Prism 6.01 | GraphPad Software, La Jolla, CA, USA |
| Image Lab ™ 5.2.1 | Bio-Rad |
| LipoP 1.0 | Juncker et al., 2003 (144) |
| Phobius | Käll et al., 2007 (146) |
| SignalBLAST | Frank et al., 2008 (147) |
| SignalP4.1 | Petersen et al., 2011 (145) |
| WebLogo 3 | Crooks et al., 2004 (178) |

TABLE 10
Machines

| Machine | Manufacturer |
|---|---|
| Anaerobic Chamber, Vinyl | Coy |
| ChemiDoc ™ Touch Imaging System | Bio-Rad |
| Gene Pulser Xcell ™ Electroporation Systems | Bio-Rad |
| iBlot ™ Gel Transfer Device | ThermoFisher Scientific |
| High-speed centrifuge Avanti J-26-S | Beckman Coulter |
| Synergy H1 Hybrid Reader | BioTek |
| TissueLyser | Qiagen |

### Methods

Bacterial Growth Conditions.

Bacterial strains used in this study are listed above. E. coli S17.1 λpir was grown in LB medium or on LB agar supplemented with carbenicillin (100 μg/mL) for plasmid selection at 37° C. All Bacteroides strains were grown in BHIS or TYG media or plates in a Coy anaerobic chamber with 85% N₂, 5% H₂, and 10% CO₂ at 37° C. Media and plates were pre-reduced overnight in an anaerobic atmosphere before culture inoculation. The antibiotics erythromycin (25 μg/mL) and gentamicin (200 μg/mL) were supplemented when necessary. Expression of fusion proteins was induced by addition of isopropyl β-D-1-thiogalactopyranoside (IPTG) (100 μM).

Generation of Recombinant Bacteroides Strains.

pNBU1 constructs were generated in E. coli and then conjugated into Bacteroides strains. For IL-10-constructs, a codon-optimized sequence for murine IL-10 was purchased as gBlocks gene fragment from Integrated DNA Technologies and PCR-amplified. OMV-tag-NL/IL-10 fusion proteins were cloned into a pNBU1 backbone by Gibson assembly. The pNBU1 plasmid encodes the intN1 tyrosine integrase, which mediates sequence-specific recombination between the attN site of pNBU1 and one of two attBT sites of the tRNA^{Ser} genes in the Bacteroides genome. Insertion inactivates the tRNA^{Ser} gene. Simultaneous insertion into both tRNA^{Ser} genes is unlikely due to the essentiality of tRNA^{Ser}.

Polymerase Chain Reaction (PCR).

Primers were designed with the software Geneious® to have 22-38 base pairs annealing with the template with melting temperatures of 55-65° C., calculated with the OligoCalc melting temperature calculator (biotools.nubic.northwestern.edu/OligoCalc.html#helpthermo) (nearest neighbor). Homology regions for Gibson assembly were added to the 5′ end where necessary. 50 μL reactions with KAPA HiFi polymerase and 1 ng template DNA for 25 cycles were performed according to the instructions of the manufacturer. Extension durations were adjusted for each fragment to be 30 s/1 kbp. Primers and plasmids used in this study are listed in above.

DNA Gel Electrophoresis and Purification.

PCR products were analyzed by gel electrophoresis (135 V, 25 min) of 5 μl PCR product in DNA gel loading dye using 1% agarose gels stained with 1×SYBR safe DNA gel stain. DNA bands were visualized with the ChemiDoc imaging system. Remaining 45 μL PCR products were purified with the QIAquick PCR purification kit. DpnI digestion of purified PCR products was performed in CutSmart buffer for 1 h at 37° C. according to the manufacturer's instruction. DNA purification was repeated and DNA was eluted in 50 μL elution buffer.

Gibson Assembly.

Gibson assembly of DNA fragments with 30-60 base pair homology regions were done in 10 μL reactions containing 5 μL house-made Gibson assembly master mix and 5 μL mix of the DNA to be assembled, where DNA fragments should be in equimolar amounts (10-100 ng of each). Reaction mix was incubated for 1 h at 50° C.

Transformation.

Electrocompetent E. coli S17.1 λpir was transformed as follows. Bacteria were thawed on ice and 2 μL Gibson assembly mix with DNA was added. Cells were transferred into a pre-cooled electroporation cuvette (0.2 cm gap) and pulsed with 2500 V in an electroporator. 1 mL LB was immediately added and cells were recovered for 1 h at 37° C. while shaking. Cells were plated on pre-warmed LB agar plates containing carbenicillin (100 μg/mL) for plasmid selection and incubated overnight at 37° C.

Plasmid Purification and Sanger DNA Sequencing.

The presence of the correctly assembled DNA construct was verified by Sanger Sequencing. Therefore, DNA of a 3 mL overnight E. coli culture was isolated using the QIAprep spin miniprep kit according to the manufacturer's protocol. Plasmids were sequenced by QuintaraBio and sequences were analyzed with Geneious.

Conjugation of Constructs into Bacteroides Spp.

Constructs were conjugated from E. coli into B. thetaiotaomicron, B. fragilis, or B. vulgatus as follows. E. coli donor and Bacteroides recipient strains were grown overnight. 250 μL E. coli culture was washed once with PBS and cell pellet was resuspended in 1 mL Bacteroides culture (1:5 ratio). The mating mixture was pelleted, resuspended in 25 μL BHIS medium, and spotted onto pre-warmed BHIS agar plates. Plates were incubated upright aerobically overnight at 37° C. On day 2, cells were scraped off from the plate and fully resuspended in 1 mL BHIS medium. 250 μL suspension was plated on BHIS agar plates containing gentamicin (200 μg/mL) for Bacteroides selection and erythromycin (25 μg/mL) for plasmid selection (BHIS+Gm+Em). Plates were incubated anaerobically for 48 h at 37° C. On day 4, colonies were re-isolated on pre-reduced BHIS+Gm+Em plates. On day 5, colonies could be used for liquid overnight cultures in BHIS or TYG medium.

Preparation of Culture Supernatants, Concentrated Supernatants, and Outer Membrane Vesicle (OMVs).

Bacteroides cultures were grown overnight and subcultured 1:100 in prereduced BHIS or TYG media supplemented with IPTG (100 μM). After 6-20 h the optical densities at 600 nm (OD600) of 300 μl of cultures were measured and proceeded as follows.

Preparation of Culture Supernatants.

NL-producing Bacteroides cultures where centrifuged at 5,000×g for 5 min. Culture supernatants were saved and cell pellets were washed in 1 mL 1× phosphate-buffered saline (PBS) and resuspended in 1 mL PBS. Luciferase activity of culture supernatant and cells was measured as described in 6.2.5.

Preparation of Concentrated Supernatants.

mIL-10 producing Bacteroides cultures where centrifuged at 10,000×g for 15 min. Phenylmethyl-sulfonyl fluoride (PMSF) (1 mM) and proteinase inhibitor cocktail was added to culture supernatants according to the manufacturer's instructions. Supernatants were concentrated 20-25-fold using centrifugal filters with 10 kDa and 100 kDa cut-off membranes according to the manufacturer's instruction with 14,000×g for concentration spin 1,000×g for reverse spin. Concentrated supernatants were stored at −80° C. at least overnight and mIL-10 concentration were determined as described above.

OMV Purification.

50-100 mL of Bacteroides subcultures were grown for 6-20 h in BHIS or TYG medium supplemented with IPTG (100 μM) and centrifuged at 10,000×g for 15 min at 4° C. PMSF (1 mM) and proteinase inhibitor cocktail was added to culture supernatants according to the manufacturer's instructions. Supernatant was filtered through a 0.45 μm membrane and centrifuged at 70,000×g for 70 min at 4° C. The pellet was washed with PBS and the centrifugation step was repeated. The OMV-containing pellet was resuspended in 100 μL PBS and stored at 4° C. until usage if necessary. NL activity or mIL-10 concentrations of purified OMVs were determined as described in 6.2.5. and 6.2.7., respectively.

Purity of OMVs was assessed by transmission electron microscopy. Samples were absorbed onto carbon-coated copper grids, washed with ddH₂O, and stained with 1% aqueous uranyl acetate. Samples were viewed on a JEOL 1200EX transmission electron microscope.

Bradford Protein Quantification Assay.

The quick start Bradford protein assay was used for colorimetric detection and quantification of protein concentrations in concentrated supernatants and OMV suspensions according to the microplate microassay protocol provided by the manufacturer. Briefly, samples were diluted in PBS according to expected protein concentration. Bovine serum albumin (BSA) was serially diluted in PBS to generate standards of known concentrations of 0-25 μg/mL. 150 μL standard or diluted sample were mixed with 150 μL of 1× dye reagent and incubated at room temperature for 5-10 min. Absorbance was measured at 595 nm on a microplate hybrid reader. Standards and samples were tested in triplicates. The concentration of each sample was determined based on the standard curve of known BSA concentrations.

Luciferase Assay.

Luciferase activities of cell suspensions, culture supernatants, or purified OMVs prepared from NL-producing Bacteroides cultures as described in 6.2.3. were determined by a Nano-Glo luciferase assay. NanoLuc luciferase present in samples catalyzes oxidation of the exogenously added substrate furimazine to generate a glow-type bioluminescence (λₘₐₓ=460 nm) with a signal half-life of ca. 120 minutes. The working reagent was prepared by diluting luciferase assay substrate 1:50 in luciferase assay buffer. The reagent contains an integral lysis buffer that allows usage directly on cells expressing NanoLuc luciferase. 25 μL working reagent was mixed with 25 μL sample (cell suspensions, culture supernatants, purified OMVs, or feces homogenate) and luminescence was measured with an integration time of 1 second at a gain setting of 100 in a microplate hybrid reader. Luciferase activities were normalized to the OD600 of 300 μl of culture at the time of harvest.

Western Blot Analysis: Sodium Dodecyl Sulfate Polyacrylamide Gel Electrophoresis (SDS-PAGE).

OMV suspensions were diluted in PBS to obtain same concentrations (as determined by Bradford assay) among all samples. 4 or 5 μg OMVs were mixed with 4×LDS sample buffer containing 10% β-mercaptoethanol, boiled for 10 minutes at 99° C., and loaded on 4-12% Bis-Tris NuPAGE gels. Proteins were separated by molecular weight at 200 V for 35 min in 1×MES SDS running buffer.

Western Blotting and Immunodetection.

Proteins were transferred onto nitrocellulose membranes by a dry electroblotting system using the iBlot™ gel transfer device and iBlot™ Gel transfer stacks with integrated nitrocellulose transfer membrane. Program P3 (20 V) was run for 6 or 7 minutes, depending on the protein size. Membranes were blocked with 1×PBS-T (0.05% Tween20 in PBS) containing 5% skim milk for at least 120 min at RT and gentle shaking. After washing with PBS-T, membranes were incubated with anti-6×His tag primary antibody (1:5,000) in PBS-T+5% milk overnight at 4° C. and gentle shaking. Membranes were then washed with PBS-T and incubated in horseradish peroxidase (HRP)-conjugated anti-mouse secondary antibody (1:5,000) in PBS-T+5% skim milk for 1 hour at RT. After washing with PBS-T, immunoreactivity was detected by the enhanced chemiluminescence (ECL) method using the Clarity™ Western ECL Substrate according the manufacturer's suggestions.

mIL-10 Quantification by Enzyme Linked Immunosorbent Assay (ELISA).

Mouse IL-10 of concentrated supernatants and OMVs was quantified by sandwich ELISA using the mouse IL-10 DuoSet ELISA kit according to the manufacturer's instructions. Recombinant mouse IL-10 standard was 2-fold serially diluted in reagent diluent (1% BSA in PBS) to generate eight standards of known concentrations of 0-2,000 pg/mL. Concentrated supernatants and OMVs were diluted 1:5 to 1:20 in reagent diluent according to expected mIL-10 concentration.

A 96-well microplate was coated with 100 μL of the α-mIL-10 capture antibody, diluted in PBS as instructed, per well and incubated overnight at room temperature. Wells were washed with 1× wash buffer (0.05% Tween20 in PBS) three times. After wash buffer was removed completely, wells were blocked by 300 μL reagent diluent for at least 60 min at room temperature and washing steps were repeated. 100 μL of sample or standards in reagent diluent were added to each well and incubated for 120 min at room temperature. After three washing steps, 100 μL of biotinylated α-mIL-10 detection antibody, diluted in reagent diluent as instructed, were added to each well and incubated for 120 min at room temperature. Microplate was washed three times, 100 μL of the working dilution of Streptavidin-HRP was added to each well, and incubated for 20 min at room temperature in the dark. After three washing steps, 100 μL of substrate solution (1:1 mixture of color reagent A (H₂O₂) and color reagent B (Tetramethylbenzidine)) per well were incubated for 20 minutes at room temperature in the dark. 50 μL of stop solution (2 N H₂SO₄) was added per well and gently mixed. Absorbance at 450 and 540 nm in each well was immediately measured using a microplate reader. Wavelength correction was done by subtracting the readings at 540 nm from the readings at 450 nm. All standards and samples were assayed in duplicates. mIL-10 concentrations in samples were determined based on the standard curve of known mIL-10 concentrations. mIL-10 concentrations were normalized to total protein concentrations as determined by Bradford assay.

Proteinase K Protection Assay.

To determine if OMV tag-NL fusion proteins are exposed on the OMV surface, accessibility to proteolytic activity was tested. Suspensions of 5 μg of OMVs in PBS were treated with 0.1 mg/mL proteinase K for various times between 30 min and 24 h at 37° C. in the presence or absence of 1% SDS (for western blot analysis) or 1% Triton-X100 (for luciferase assay). Following the incubation, all samples were placed on ice and proteolysis was stopped by addition of 1 mM phenylmethanesulfonyl fluoride (PMSF) when analyzed by western blot. The effects of proteinase K and detergents treatments on OMV-tag-NL loaded OMVs were determined by luciferase assay or western blot.

### Mouse Studies

Animal study protocols were approved by the MIT Animal Care and Use Committee. 8-10 weeks-old male C57BL/6 mice were housed in non-sterile conditions with access to irradiated mouse chow and autoclaved water. 10 days prior to gavage of Bacteroides spp., mice were administered ciprofloxacin HCl (0.625 g/L) in sugar-sweetened drinking water and treated with metronidazole (1 mg/kg) by oral gavage every 24 h for 7 days. After antibiotic medication was stopped for 2 days, engineered Bacteroides spp. (5×10⁸ CFU in 0.1 mL of 20% sucrose) were administered by oral gavage. Groups of mice (n=5) received either B. thetaiotaomicron, B. fragilis, B. vulgatus, or no bacteria. On day 4 or 7 after bacterial gavage acute colitis was induced by administration of 3% (w/v) dextran sulfate sodium (DSS, molecular weight 40-50 kDa) in drinking water for 8 days. Simultaneously, IPTG (25 mM) was administered in drinking water to induce the expression of the recombinant protein. Mice belonging to the same group were co-housed for the duration of the experiment. Body weight was recorded daily for the duration of the experiment. At the end of the study, mice were sacrificed and colonic length was measured, colon samples were taken for histological analysis, and fecal pellets were collected.

CFU and Luminescence in Feces.

Feces were weighted, 1 mL PBS was added, and were homogenized in a TissueLyser for 2 min at 25 Hz using sterile steel beads. Mixture was spun (30 s, 500×g) to pellet large debris. 1:10 serial dilutions were plated on selective BHIS+Gm+Em agar plates in six technical replicates. Plates were incubated anaerobically for 48 h at 37° C. and colony-forming units (CFU) were count. For measurement of luciferase activity, 25 μL of feces homogenate was mixed with 25 μL NanoLuc working solution and luminescence was read in a hybrid microplate reader as described above.

Histological Analysis.

Colon tissue was fixed in 10% (w/v) formalin, paraffin-embedded, sectioned, and stained with haematoxylin & eosin (H&E). Three sections (proximal, mid, and distal colon) per animal were microscopically scored on a scale of 0-4 with 0.5 increments for the following 7 criteria: inflammatory infiltrates, edema, epithelial defects, extent of epithelial defects/crypt atrophy, epithelial hyperplasia, dysplasia/neoplasia, and area of dysplasia/neoplasia. The total colitis score is the sum of the 7 sub-scores. Samples were read by a pathologist blinded to the identity of the samples.

### Bioinformatics Analysis

The presence of N-terminal signal peptides and SPII cleavage sites were predicted with LipoP 1.0 (cbs.dtu.dk/services/LipoP/) (144), SignalP 4.1 (cbs.dtu.dk/services/SignalP/) (145), SignalBLAST (sigpep.services.came.sbg.ac.at/signalblast.html) (147), and Phobius (phobius.sbc.su.se/) (146) using default settings for Gram negative bacteria. Only LipoP discriminates between signal peptides of secreted proteins and lipoproteins as well as N-terminal transmembrane helices in Gram-negative bacteria (144). SignalBLAST predicts signal peptides based on sequence alignment techniques.

The UniProt database (uniprot.org/) (148) was used to search for ‘lipoprotein’ in Bacteroides thetaiotaomicron (strain ATCC 29148/DSM 2079/NCTC 10582/E50/VPI-5482). Of the 63 obtained results, only proteins that possessed lipoprotein signal peptides predicted by LipoP and were not detected in OMVs by Elhenawy et al. (74) were used for further analysis.

Protein sequence alignments were carried out using ClustalOmega (ebi.ac.uk/Tools/msa/clustalo/) or T-Coffee (ebi.ac.uk/Tools/msa/tcoffee/) software.

Sequence logos of 4 aa before and 6 aa after the SPII cleavage site as predicted by LipoP were generated using the WebLogo 3 software (weblogo.threeplusone.com/create.cgi) (178).

### Data Analysis

Data were analyzed using the GraphPad Prism 6.01 software and are presented as mean values±standard deviation (SD). Statistical significance was analyzed by two-tailed Student's t-tests with 95% confidence interval. The level of significance was set to p<0.05 for all experiments.

### Abbreviations

aa amino acid
Bf B. fragilis
BHIS brain-heart infusion
Bt B. thetaiotaomicron
Bvm B. vulgatus mouse isolate
ccf commensal colonization factors
CD Crohn's disease
CFU colony-forming units
DC dendritic cell
DSS dextran sulfate sodium
ELISA enzyme-linked immunosorbent assay
Em erythromycin
FL full length
Gm gentamicin
IBD inflammatory bowel diseases
IL interleukin
IM inner membrane
InsP6 Inositol hexakisphosphate
IPTG isopropyl β-D-1-thiogalactopyranoside
KGF keratinocyte growth factor
lpp Braun's lipoprotein
LPS lipopolysaccharide
NK natural killer
NL NanoLuc
NO nitric oxide
OM outer membrane
OMV outer membrane vesicle
OMV proteins BT1491, BT3238, BACOVA_04502
OMV tags BT1491Δ25, BT3238Δ35, BACOVA_04502Δ28
PG peptidoglycan
PK proteinase K
PMSF phenylmethylsulfonyl fluoride
PSA polysaccharide A
RLU relative light units
SPF Specific-pathogen-free
SPI/II signal peptidase I/II
TGF transforming growth factor
Th T helper cell
TNF tumor necrosis factor
Tregs regulatory T cells
TYG trypticase yeast extract glucose
UC ulcerative colitis

### REFERENCES

1. R. Berg, The indigenous gastrointestinal microflora. Trends Microbiol. 4, 430-5 (1996).
2. D. C. Savage, Microbial ecology of the gastrointestinal tract. Annu. Rev. Microbiol. 31, 107-33 (1977).
3. R. Sender, S. Fuchs, R. Milo, Revised estimates for the number of human and bacteria cells in the body. PLOS Biol., 1-21 (2016).
4. J. Xu, J. I. Gordon, Honor thy symbionts. Proc. Natl. Acad. Sci. U.S.A. 100, 10452-9 (2003).
5. M. Breitbart et al., Metagenomic analyses of an uncultured viral community from human feces. J. Bacteriol. 185, 6220-3 (2003).
6. P. B. Eckburg, P. W. Lepp, D. A. Relman, Archaea and their potential role in human disease. Infect. Immun. 71, 591-6 (2003).
7. C. A. Lozupone, J. I. Stombaugh, J. I. Gordon, J. K. Jansson, R. Knight, Diversity, stability and resilience of the human gut microbiota. Nature. 489, 220-230 (2012).
8. W. E. Moore, L. V Holdeman, Human fecal flora: the normal flora of 20 Japanese-Hawaiians. Appl. Microbiol. 27, 961-79 (1974).
9. F. Bäckhed, R. E. Ley, J. L. Sonnenburg, D. A. Peterson, J. I. Gordon, Host-bacterial mutualism in the human intestine. Science. 307, 1915-20 (2005).
10. V. Bocci, The neglected organ: bacterial flora has a crucial immunostimulatory role. Perspect. Biol. Med. 35, 251-60 (1992).
11. E. G. Zoetendal, A. D. Akkermans, W. M. De Vos, Temperature gradient gel electrophoresis analysis of 16S rRNA from human fecal samples reveals stable and host-specific communities of active bacteria. Appl. Environ. Microbiol. 64, 3854-9 (1998).
12. C. L. Sears, A dynamic partnership: Celebrating our gut flora. Anaerobe. 11, 247-251 (2005).
13. A. M. O'Hara, F. Shanahan, The gut flora as a forgotten organ. EMBO Rep. 7, 688-93 (2006).
14. L. V Hooper, T. Midtvedt, J. I. Gordon, How host-microbial interactions shape the nutrient environment of the mammalian intestine. Annu. Rev. Nutr. 22, 283-307 (2002).
15. E. N. Bergman, Energy contributions of volatile fatty acids from the gastrointestinal tract in various species. Physiol. Rev. 70, 567-590 (1990).
16. P. B. Hylemon, J. Harder, Biotransformation of monoterpenes, bile acids, and other isoprenoids in anaerobic ecosystems. FEMS Microbiol. Rev. 22, 475-88 (1998).
17. M. J. Hill, Intestinal flora and endogenous vitamin synthesis. Eur. J. Cancer Prey. 6 Suppl 1, S43-5 (1997).
18. C. L. Wells, M. A. Maddaus, R. P. Jechorek, R. L. Simmons, Role of intestinal anaerobic bacteria in colonization resistance. Eur. J. Clin. Microbiol. Infect. Dis. 7, 107-13 (1988).
19. H. Ashida, M. Ogawa, M. Kim, H. Mimuro, C. Sasakawa, Bacteria and host interactions in the gut epithelial barrier. Nat. Chem. Biol. 8, 36-45 (2011).
20. N. Kamada, G. Y. Chen, N. Inohara, G. Nunez, Control of pathogens and pathobionts by the gut microbiota. Nat. Immunol. 14, 685-90 (2013).
21. C. Braun-Fahrländer et al., Environmental Exposure to Endotoxin and Its Relation to Asthma in School-Age Children. N. Engl. J. Med. 347, 869-877 (2002).
22. A. T. Stefka et al., Commensal bacteria protect against food allergen sensitization. Proc. Natl. Acad. Sci. 111, 13145-13150 (2014).
23. J. Claesen, M. A. Fischbach, Synthetic microbes as drug delivery systems. ACS Synth. Biol. 4, 358-64 (2015).
24. M. Mimee, R. J. Citorik, T. K. Lu, Microbiome therapeutics—Advances and challenges. Adv. Drug Deliv. Rev. 105, 44-54 (2016).
25. L. V Holdeman, I. J. Good, W. E. Moore, Human fecal flora: variation in bacterial composition within individuals and a possible effect of emotional stress. Appl. Environ. Microbiol. 31, 359-75 (1976).
26. A. A. Salyers, Bacteroides of the human lower intestinal tract. Annu. Rev. Microbiol. 38, 293-313 (1984).
27. J. Xu et al., A genomic view of the human-Bacteroides thetaiotaomicron symbiosis. Science. 299, 2074-6 (2003).
28. S. M. Lee et al., Bacterial colonization factors control specificity and stability of the gut microbiota. Nature. 501, 426-9 (2013).
29. L. Pumbwe, C. A. Skilbeck, H. M. Wexler, Impact of anatomic site on growth, efflux-pump expression, cell structure, and stress responsiveness of Bacteroides fragilis. Curr. Microbiol. 55, 362-5 (2007).
30. J. J. Faith et al., The Long-Term Stability of the Human Gut Microbiota. Science (80-.). 341, 480-484 (2013).
31. H. M. Wexler, Bacteroides: the good, the bad, and the nitty-gritty. Clin. Microbiol. Rev. 20, 593-621 (2007).
32. B. F. Polk, D. L. Kasper, Bacteroides fragilis subspecies in clinical isolates. Ann. Intern. Med. 86, 569-71 (1977).
33. S. K. Mazmanian, C. H. Liu, A. O. Tzianabos, D. L. Kasper, An immunomodulatory molecule of symbiotic bacteria directs maturation of the host immune system. Cell. 122, 107-18 (2005).
34. S. K. Mazmanian, J. L. Round, D. L. Kasper, A microbial symbiosis factor prevents intestinal inflammatory disease. Nature. 453, 620-625 (2008).
35. J. L. Round, S. K. Mazmanian, Inducible Foxp3+ regulatory T-cell development by a commensal bacterium of the intestinal microbiota. Proc. Natl. Acad. Sci. U.S.A. 107, 12204-9 (2010).
36. H. L. Cash, C. V Whitham, C. L. Behrendt, L. V Hooper, Symbiotic bacteria direct expression of an intestinal bactericidal lectin. Science. 313, 1126-30 (2006).
37. L. V. Hooper, T. S. Stappenbeck, C. V. Hong, J. I. Gordon, Angiogenins: a new class of microbicidal proteins involved in innate immunity. Nat. Immunol. 4, 269-273 (2003).
38. E. Sefik et al., MUCOSAL IMMUNOLOGY. Individual intestinal symbionts induce a distinct population of RORγ⁺regulatory T cells. Science. 349, 993-7 (2015).
39. G. T. MacFarlane, G. R. Gibson, Co-utilization of polymerized carbon sources by Bacteroides ovatus grown in a two-stage continuous culture system. Appl. Environ. Microbiol. 57, 1-6 (1991).
40. A. A. Salyers, S. E. West, J. R. Vercellotti, T. D. Wilkins, Fermentation of mucins and plant polysaccharides by anaerobic bacteria from the human colon. Appl. Environ. Microbiol. 34, 529-33 (1977).
41. A. A. Salyers, J. R. Vercellotti, S. E. West, T. D. Wilkins, Fermentation of mucin and plant polysaccharides by strains of Bacteroides from the human colon. Appl. Environ. Microbiol. 33, 319-22 (1977).
42. M. K. Bjursell, E. C. Martens, J. I. Gordon, Functional genomic and metabolic studies of the adaptations of a prominent adult human gut symbiont, Bacteroides thetaiotaomicron, to the suckling period. J. Biol. Chem. 281, 36269-79 (2006).
43. J. L. Sonnenburg, L. T. Angenent, J. I. Gordon, Getting a grip on things: how do communities of bacterial symbionts become established in our intestine? Nat. Immunol. 5, 569-573 (2004).
44. S. Rakoff-Nahoum, K. R. Foster, L. E. Comstock, The evolution of cooperation within the gut microbiota. Nature. 533, 255-259 (2016).
45. T.-T. Tseng et al., Protein secretion systems in bacterial-host associations, and their description in the Gene Ontology. BMC Microbiol. 9, S2 (2009).
46. A. Kulp, M. J. Kuehn, Biological functions and biogenesis of secreted bacterial outer membrane vesicles. Annu Rev Microbiol. 64, 163-184 (2010).
47. H. M. Kulkarni, M. V. Jagannadham, Biogenesis and multifaceted roles of outer membrane vesicles from Gram-negative bacteria. Microbiol. (United Kingdom). 160, 2109-2121 (2014).
48. C. Schwechheimer, M. J. Kuehn, Outer-membrane vesicles from Gram-negative bacteria: biogenesis and functions. Nat. Rev. Microbiol. 13, 605-19 (2015).
49. R. C. Alaniz, B. L. Deatherage, J. C. Lara, B. T. Cookson, Membrane Vesicles Are Immunogenic Facsimiles of Salmonella typhimurium That Potently Activate Dendritic Cells, Prime B and T Cell Responses, and Stimulate Protective Immunity In Vivo. J. Immunol. 179, 7692-7701 (2007).
50. T. N. Ellis, M. J. Kuehn, Virulence and immunomodulatory roles of bacterial outer membrane vesicles. Microbiol. Mol. Biol. Rev. 74, 81-94 (2010).
51. S. J. Biller et al., Bacterial vesicles in marine ecosystems. Science. 343, 183-6 (2014).
52. T. J. Beveridge, S. A. Makin, J. L. Kadurugamuwa, Z. Li, Interactions between biofilms and the environment. FEMS Microbiol. Rev. 20, 291-303 (1997).
53. L. M. Mashburn-Warren, M. Whiteley, Special delivery: vesicle trafficking in prokaryotes. Mol. Microbiol. 61, 839-46 (2006).
54. T. J. Silhavy, D. Kahne, S. Walker, The bacterial cell envelope. Cold Spring Harb. Perspect. Biol. 2, a000414 (2010).
55. N. Katsui et al., Heat-induced blebbing and vesiculation of the outer membrane of Escherichia coli. J. Bacteriol. 151, 1523-31 (1982).
56. A. J. McBroom, M. J. Kuehn, Release of outer membrane vesicles by Gram-negative bacteria is a novel envelope stress response. Mol. Microbiol. 63, 545-58 (2007).
57. B. van de Waterbeemd et al., Cysteine depletion causes oxidative stress and triggers outer membrane vesicle release by Neisseria meningitidis; implications for vaccine development. PLoS One. 8, e54314 (2013).
58. J. L. Kadurugamuwa, T. J. Beveridge, Virulence factors are released from Pseudomonas aeruginosa in association with membrane vesicles during normal growth and exposure to gentamicin: a novel mechanism of enzyme secretion. J. Bacteriol. 177, 3998-4008 (1995).
59. C. Schwechheimer, C. J. Sullivan, M. J. Kuehn, Envelope control of outer membrane vesicle production in Gram-negative bacteria. Biochemistry. 52, 3031-40 (2013).
60. C. Schwechheimer, D. L. Rodriguez, M. J. Kuehn, NlpI-mediated modulation of outer membrane vesicle production through peptidoglycan dynamics in Escherichia coli. Microbiologyopen. 4, 375-89 (2015).
61. J. Wensink, B. Witholt, Outer-membrane vesicles released by normally growing Escherichia coli contain very little lipoprotein. Eur. J. Biochem. 116, 331-5 (1981).
62. C. Schwechheimer, A. Kulp, M. J. Kuehn, Modulation of bacterial outer membrane vesicle production by envelope structure and content. BMC Microbiol. 14, 324 (2014).
63. Y. Tashiro et al., Outer membrane machinery and alginate synthesis regulators control membrane vesicle production in Pseudomonas aeruginosa. J. Bacteriol. 191, 7509-19 (2009).
64. M. Kaparakis et al., Bacterial membrane vesicles deliver peptidoglycan to NOD1 in epithelial cells. Cell. Microbiol. 12, 372-385 (2010).
65. Z. Li, A. J. Clarke, T. J. Beveridge, A major autolysin of Pseudomonas aeruginosa: subcellular distribution, potential role in cell growth and division and secretion in surface membrane vesicles. J. Bacteriol. 178, 2479-88 (1996).
66. J. W. Schertzer, M. Whiteley, A Bilayer-Couple Model of Bacterial Outer Membrane Vesicle Biogenesis. MBio. 3, e00297-11-e00297-11 (2012).
67. A. J. McBroom, A. P. Johnson, S. Vemulapalli, M. J. Kuehn, Outer membrane vesicle production by Escherichia coli is independent of membrane instability. J. Bacteriol. 188, 5385-92 (2006).
68. E.-Y. Lee, D.-S. Choi, K.-P. Kim, Y. S. Gho, Proteomics in gram-negative bacterial outer membrane vesicles. Mass Spectrom. Rev. 27, 535-555 (2008).
69. D. Chatterjee, K. Chaudhuri, Association of cholera toxin with Vibrio cholerae outer membrane vesicles which are internalized by human intestinal epithelial cells. FEBS Lett. 585, 1357-1362 (2011).
70. S. N. Wai et al., Vesicle-Mediated Export and Assembly of Pore-Forming Oligomers of the Enterobacterial ClyA Cytotoxin. Cell. 115, 25-35 (2003).
71. S. J. Bauman, M. J. Kuehn, Purification of outer membrane vesicles from Pseudomonas aeruginosa and their activation of an IL-8 response. Microbes Infect. 8, 2400-8 (2006).
72. S. Kato, Y. Kowashi, D. R. Demuth, Outer membrane-like vesicles secreted by Actinobacillus actinomycetemcomitans are enriched in leukotoxin. Microb. Pathog. 32, 1-13 (2002).
73. E.-Y. Lee et al., Global proteomic profiling of native outer membrane vesicles derived from Escherichia coli. Proteomics. 7, 3143-53 (2007).
74. W. Elhenawy, M. O. Debelyy, M. F. Feldman, Preferential Packing of Acidic Glycosidases and Proteases into Bacteroides Outer Membrane Vesicles. MBio. 5, e00909-14-e00909-14 (2014).
75. K. E. Bonnington, M. J. Kuehn, Protein selection and export via outer membrane vesicles. Biochim. Biophys. Acta. 1843, 1612-9 (2014).
76. M. Muller, H. G. Koch, K. Beck, U. Schäfer, Protein traffic in bacteria: multiple routes from the ribosome to and across the membrane. Prog. Nucleic Acid Res. Mol. Biol. 66, 107-57 (2001).
77. H. Mori, K. Ito, The Sec protein-translocation pathway. Trends Microbiol. 9, 494-500 (2001).
78. A. J. Driessen, E. H. Manting, C. van der Does, The structural basis of protein targeting and translocation in bacteria. Nat. Struct. Biol. 8, 492-8 (2001).
79. C. L. Santini et al., A novel sec-independent periplasmic protein translocation pathway in Escherichia coli. EMBO J. 17, 101-12 (1998).
80. P. A. Lee, D. Tullman-Ercek, G. Georgiou, The bacterial twin-arginine translocation pathway. Annu. Rev. Microbiol. 60, 373-95 (2006).
81. J. G. Sklar et al., Lipoprotein SmpA is a component of the YaeT complex that assembles outer membrane proteins in Escherichia coli. Proc. Natl. Acad. Sci. U.S.A. 104, 6400-5 (2007).
82. S. Kim et al., Structure and function of an essential component of the outer membrane protein assembly machine. Science. 317, 961-4 (2007).
83. F. J. del Castillo, S. C. Leal, F. Moreno, I. del Castillo, The Escherichia coli K-12 sheA gene encodes a 34-kDa secreted haemolysin. Mol. Microbiol. 25, 107-15 (1997).
84. J.-Y. Kim et al., Engineered bacterial outer membrane vesicles with enhanced functionality. J. Mol. Biol. 380, 51-66 (2008).
85. J. L. Kadurugamuwa, T. J. Beveridge, Bacteriolytic effect of membrane vesicles from Pseudomonas aeruginosa on other bacteria including pathogens: conceptually new antibiotics. J. Bacteriol. 178, 2767-74 (1996).
86. Z. Li, A. J. Clarke, T. J. Beveridge, Gram-negative bacteria produce membrane vesicles which are capable of killing other bacteria. J. Bacteriol. 180, 5478-83 (1998).
87. E. J. O'Donoghue, A. M. Krachler, Mechanisms of outer membrane vesicle entry into host cells. Cell. Microbiol. (2016), doi:10.1111/cmi.12655.
88. J. L. Kadurugamuwa, T. J. Beveridge, Delivery of the non-membrane-permeative antibiotic gentamicin into mammalian cells by using Shigella flexneri membrane vesicles. Antimicrob. Agents Chemother. 42, 1476-83 (1998).
89. J. M. Bomberger et al., Long-distance delivery of bacterial virulence factors by Pseudomonas aeruginosa outer membrane vesicles. PLoS Pathog. 5, e1000382 (2009).
90. M. Kaparakis-Liaskos, R. L. Ferrero, Immune modulation by bacterial outer membrane vesicles. Nat. Rev. Immunol. 15, 375-387 (2015).
91. V. Schaar et al., Multicomponent Moraxella catarrhalis outer membrane vesicles induce an inflammatory response and are internalized by human epithelial cells. Cell. Microbiol. 13, 432-49 (2011).
92. S. K. Vanaja et al., Bacterial Outer Membrane Vesicles Mediate Cytosolic Localization of LPS and Caspase-11 Activation. Cell. 165, 1106-1119 (2016).
93. L. Kunsmann et al., Virulence from vesicles: Novel mechanisms of host cell injury by Escherichia coli O104:H4 outbreak strain. Sci. Rep. 5, 13252 (2015).
94. H. Parker, K. Chitcholtan, M. B. Hampton, J. I. Keenan, Uptake of Helicobacter pylori outer membrane vesicles by gastric epithelial cells. Infect. Immun. 78, 5054-61 (2010).
95. S. W. Sharpe, M. J. Kuehn, K. M. Mason, Elicitation of epithelial cell-derived immune effectors by outer membrane vesicles of nontypeable Haemophilus influenzae. Infect. Immun. 79, 4361-9 (2011).
96. J. Berleman, M. Auer, The role of bacterial outer membrane vesicles for intra- and interspecies delivery. Environ. Microbiol. 15, 347-354 (2013).
97. B. L. Deatherage, B. T. Cookson, Membrane vesicle release in bacteria, eukaryotes, and archaea: a conserved yet underappreciated aspect of microbial life. Infect. Immun. 80, 1948-57 (2012).
98. Y. Shen et al., Outer membrane vesicles of a human commensal mediate immune regulation and disease protection. Cell Host Microbe. 12, 509-20 (2012).
99. R. Stentz et al., A bacterial homolog of a eukaryotic inositol phosphate signaling enzyme mediates cross-kingdom dialog in the mammalian gut. Cell Rep. 6, 646-56 (2014).
100. A. J. Manning, M. J. Kuehn, Contribution of bacterial outer membrane vesicles to innate bacterial defense. BMC Microbiol. 11, 258 (2011).
101. O. Ciofu, T. J. Beveridge, J. Kadurugamuwa, J. Walther-Rasmussen, N. Høiby, Chromosomal beta-lactamase is packaged into membrane vesicles and secreted from Pseudomonas aeruginosa. J. Antimicrob. Chemother. 45, 9-13 (2000).
102. M. R. Loeb, J. Kilner, Release of a special fraction of the outer membrane from both growing and phage T4-infected Escherichia coli B. Biochim. Biophys. Acta-Biomembr. 514, 117-127 (1978).
103. L. M. Mashburn, M. Whiteley, Membrane vesicles traffic signals and facilitate group activities in a prokaryote. Nature. 437, 422-5 (2005).
104. C. A. Hickey et al., Colitogenic Bacteroides thetaiotaomicron antigens access host immune cells in a sulfatase-dependent manner via outer membrane vesicles. Cell Host Microbe. 17, 672-680 (2015).
105. N. C. Kesty, M. J. Kuehn, Incorporation of Heterologous Outer Membrane and Periplasmic Proteins into Escherichia coli Outer Membrane Vesicles. J. Biol. Chem. 279, 2069-2076 (2004).
106. C. Arigita et al., Stability of mono- and trivalent meningococcal outer membrane vesicle vaccines. Vaccine. 22, 629-42 (2004).
107. D. K. Podolsky, Inflammatory Bowel Disease. N. Engl. J. Med. 325, 928-937 (1991).
108. M. G. Neuman et al., Inflammatory bowel disease: role of diet, microbiota, life style. Transl. Res. 160, 29-44 (2012).
109. W. Strober, I. J. Fuss, Proinflammatory cytokines in the pathogenesis of inflammatory bowel diseases. Gastroenterology. 140, 1756-67 (2011).
110. C. Abraham, J. H. Cho, Inflammatory bowel disease. N. Engl. J. Med. 361, 2066-78 (2009).
111. J. R. Maxwell, W. A. Brown, C. L. Smith, F. R. Byrne, J. L. Viney, Methods of inducing inflammatory bowel disease in mice. Curr. Protoc. Pharmacol. Chapter 5, Unit5.58 (2009).
112. A. Franke et al., Sequence variants in IL10, ARPC2 and multiple other loci contribute to ulcerative colitis susceptibility. Nat. Genet. 40, 1319-23 (2008).
113. A. Franke et al., Replication of signals from recent studies of Crohn's disease identifies previously unknown disease loci for ulcerative colitis. Nat. Genet. 40, 713-5 (2008).
114. L. Jostins et al., Host-microbe interactions have shaped the genetic architecture of inflammatory bowel disease. Nature. 491, 119-24 (2012).
115. A. H. Wang et al., The effect of IL-10 genetic variation and interleukin 10 serum levels on Crohn's disease susceptibility in a New Zealand population. Hum. Immunol. 72, 431-5 (2011).
116. G. P. Aithal et al., Role of polymorphisms in the interleukin-10 gene in determining disease susceptibility and phenotype in inflamatory bowel disease. Dig. Dis. Sci. 46, 1520-5 (2001).
117. G. Roda, B. Jharap, N. Neeraj, J.-F. Colombel, Loss of Response to Anti-TNFs: Definition, Epidemiology, and Management. Clin. Transl. Gastroenterol. 7, e135 (2016).
118. G. J. Marlow, D. van Gent, L. R. Ferguson, Why interleukin-10 supplementation does not work in Crohn's disease patients. World J. Gastroenterol. 19, 3931-41 (2013).
119. W. R. Connell, M. A. Kamm, J. K. Ritchie, J. E. Lennard-Jones, Bone marrow toxicity caused by azathioprine in inflammatory bowel disease: 27 years of experience. Gut. 34, 1081-5 (1993).
120. K. W. Moore, R. de Waal Malefyt, R. L. Coffman, A. O'Garra, Interleukin-10 and the interleukin-10 receptor. Annu. Rev. Immunol. 19, 683-765 (2001).
121. R. P. Donnelly, H. Dickensheets, D. S. Finbloom, The interleukin-10 signal transduction pathway and regulation of gene expression in mononuclear phagocytes. J. Interferon Cytokine Res. 19, 563-73 (1999).
122. P. J. Murray, Understanding and exploiting the endogenous interleukin-10/STAT3-mediated anti-inflammatory response. Curr. Opin. Pharmacol. 6, 379-86 (2006).
123. T. A. Grool et al., Anti-inflammatory effect of interleukin-10 in rabbit immune complex-induced colitis. Scand. J. Gastroenterol. 33, 754-8 (1998).
124. M. Tomoyose, K. Mitsuyama, H. Ishida, A. Toyonaga, K. Tanikawa, Role of interleukin-10 in a murine model of dextran sulfate sodium-induced colitis. Scand. J. Gastroenterol. 33, 435-40 (1998).
125. G. Barbara, Z. Xing, C. M. Hogaboam, J. Gauldie, S. M. Collins, Interleukin 10 gene transfer prevents experimental colitis in rats. Gut. 46, 344-9 (2000).
126. H. Herfarth, J. Scholmerich, IL-10 therapy in Crohn's disease: at the crossroads. Treatment of Crohn's disease with the anti-inflammatory cytokine interleukin 10. Gut. 50, 146-7 (2002).
127. L. Steidler et al., Treatment of Murine Colitis by Lactococcus lactis Secreting Interleukin-10. Science (80-.). 289, 1352-1355 (2000).
128. H. Braat et al., A Phase I Trial With Transgenic Bacteria Expressing Interleukin-10 in Crohn's Disease. Clin. Gastroenterol. Hepatol. 4, 754-759 (2006).
129. Z. Z. R. Hamady et al., Xylan-regulated delivery of human keratinocyte growth factor-2 to the inflamed colon by the human anaerobic commensal bacterium Bacteroides ovatus. Gut. 59, 461-9 (2010).
130. Z. Z. R. Hamady, Novel xylan-controlled delivery of therapeutic proteins to inflamed colon by the human anaerobic commensal bacterium. Ann. R. Coll. Surg. Engl. 95, 235-40 (2013).
131. F. Duan, K. L. Curtis, J. C. March, Secretion of insulinotropic proteins by commensal bacteria: rewiring the gut to treat diabetes. Appl. Environ. Microbiol. 74, 7437-8 (2008).
132. K. Vandenbroucke et al., Orally administered L. lactis secreting an anti-TNF Nanobody demonstrate efficacy in chronic colitis. Mucosal Immunol. 3, 49-56 (2010).
133. J. L. Round, S. K. Mazmanian, The gut microbiota shapes intestinal immune responses during health and disease. Nat Rev Immunol. 9, 313-323 (2009).
134. M. D. Farrar et al., Engineering of the gut commensal bacterium Bacteroides ovatus to produce and secrete biologically active murine interleukin-2 in response to xylan. J. Appl. Microbiol. 98, 1191-1197 (2005).
135. A. Focareta, J. C. Paton, R. Morona, J. Cook, A. W. Paton, A Recombinant Probiotic for Treatment and Prevention of Cholera. Gastroenterology. 130, 1688-1695 (2006).
136. N. Saeidi et al., Engineering microbes to sense and eradicate Pseudomonas aeruginosa, a human pathogen. Mol. Syst. Biol. 7, 521 (2011).
137. P. B. Eckburg et al., Diversity of the human intestinal microbial flora. Science. 308, 1635-8 (2005).
138. M. H. Daleke-Schermerhorn et al., Decoration of outer membrane vesicles with multiple antigens by using an autotransporter approach. Appl. Environ. Microbiol. 80, 5854-5865 (2014).
139. M. Muralinath, M. J. Kuehn, K. L. Roland, R. Curtiss, Immunization with Salmonella enterica serovar typhimurium-derived outer membrane vesicles delivering the pneumococcal protein PspA confers protection against challenge with Streptococcus pneumoniae. Infect. Immon. 79, 887-894 (2011).
140. M. Mimee, A. C. Tucker, C. A. Voigt, T. K. Lu, Programming a Human Commensal Bacterium, Bacteroides thetaiotaomicron, to Sense and Respond to Stimuli in the Murine Gut Microbiota. Cell Syst. 1, 62-71 (2015).
141. S. Rakoff-Nahoum, M. J. Coyne, L. E. Comstock, An ecological network of polysaccharide utilization among human intestinal symbionts. Curr. Biol. 24, 40-9 (2014).
142. J. L. Gardy, F. S. L. Brinkman, Methods for predicting bacterial protein subcellular localization. Nat. Rev. Microbiol. 4, 741-751 (2006).
143. O. Emanuelsson, S. Brunak, G. von Heijne, H. Nielsen, Locating proteins in the cell using TargetP, SignalP and related tools. Nat. Protoc. 2, 953-971 (2007).
144. A. S. Juncker et al., Prediction of lipoprotein signal peptides in Gram-negative bacteria. Protein Sci. 12, 1652-62 (2003).
145. T. N. Petersen, S. Brunak, G. von Heijne, H. Nielsen, SignalP 4.0: discriminating signal peptides from transmembrane regions. Nat. Methods. 8, 785-786 (2011).
146. L. Käll, A. Krogh, E. L. L. Sonnhammer, Advantages of combined transmembrane topology and signal peptide prediction—the Phobius web server. Nucleic Acids Res. 35, W429-32 (2007).
147. K. Frank, M. J. Sippl, High-performance signal peptide prediction based on sequence alignment techniques. Bioinformatics. 24, 2172-2176 (2008).
148. A. Bairoch et al., The Universal Protein Resource (UniProt). Nucleic Acids Res. 33, D154-9 (2005).
149. Yamaguchi Kl, Yu F, Inouye M, A single amino acid determinant of the membrane localization of lipoproteins in E. coli. Cell. 53, 423-32 (1988).
150. H. Tokuda, S. Matsuyama, Sorting of lipoproteins to the outer membrane in E. coli. Biochim. Biophys. Acta-Mol. Cell Res. 1693, 5-13 (2004).
151. M. M. Wilson, D. E. Anderson, H. D. Bernstein, Analysis of the outer membrane proteome and secretome of Bacteroides fragilis reveals a multiplicity of secretion mechanisms. PLoS One. 10, e0117732 (2015).
152. J. L. Babb, C. S. Cummins, Encapsulation of Bacteroides Species. Infect. Immun. 19, 1088-1091 (1978).
153. R. Duchmann, E. Schmitt, P. Knolle, K. H. Meyer zum Büschenfelde, M. Neurath, Tolerance towards resident intestinal flora in mice is abrogated in experimental colitis and restored by treatment with interleukin-10 or antibodies to interleukin-12. Eur. J. Immunol. 26, 934-8 (1996).
154. W. Michael, J. Natalie, M. M. Fort, M. Donna, The Role of IL-10 in Inflammatory Bowel Disease: “Of Mice and Men”*, 123-133.
155. S. M. Bloom et al., Commensal Bacteroides species induce colitis in host-genotype-specific fashion in a mouse model of inflammatory bowel disease. Cell Host Microbe. 9, 390-403 (2011).
156. S. Wirtz, C. Neufert, B. Weigmann, M. F. Neurath, Chemically induced mouse models of intestinal inflammation. Nat. Protoc. 2, 541-6 (2007).
157. B. Chassaing, J. D. Aitken, M. Malleshappa, M. Vijay-Kumar, Curr. Protoc. Immunol., in press, doi:10.1002/0471142735.im1525s104.
158. J. Schroeder, T. Aebischer, Recombinant outer membrane vesicles to augment antigen-specific live vaccine responses. Vaccine. 27, 6748-6754 (2009).
159. J. E. Galen et al., Adaptation of the endogenous Salmonella enterica serovar Typhi clyA-encoded hemolysin for antigen export enhances the immunogenicity of anthrax protective antigen domain 4 expressed by the attenuated live-vector vaccine strain CVD 908-htrA. Infect. Immun. 72, 7096-106 (2004).
160. D. J. Chen et al., Delivery of foreign antigens by engineered outer membrane vesicle vaccines. Proc. Natl. Acad. Sci. U.S.A. 107, 3099-104 (2010).
161. S. Okuda, H. Tokuda, Lipoprotein Sorting in Bacteria. Annu. Rev. Microbiol. 65, 239-259 (2011).
162. J. M. Gennity, M. Inouye, The protein sequence responsible for lipoprotein membrane localization in Escherichia coli exhibits remarkable specificity. J. Biol. Chem. 266, 16458-16464 (1991).
163. A. P. Pugsley, M. G. Kornacker, Secretion of the cell surface lipoprotein pullulanase in Escherichia coli. J Biol Chem. 266, 13640-13645 (1991).
164. J. P. a P. Bouvier, P. Stragier, A gene for a new lipoprotein in the dapA-purC inteval of the Escherichia coli chromosome. J Bacteriol. 173, 5523-5531 (1991).
165. D. Seiffer, J. R. Klein, R. Plapp, EnvC, a new lipoprotein of the cytoplasmic membrane of Escherichia coli. FEMS Microbiol. Lett. 107, 175-8 (1993).
166. A. Seydel, P. Gounon, A. P. Pugsley, Testing the “+2 rule” for lipoprotein sorting in the Escherichia coli cell envelope with a new genetic selection. Mol. Microbiol. 34, 810-821 (1999).
167. M. Terada, T. Kuroda, S. I. Matsuyama, H. Tokuda, Lipoprotein Sorting Signals Evaluated as the LolA-dependent Release of Lipoproteins from the Cytoplasmic Membrane of Escherichia coli. J. Biol. Chem. 276, 47690-47694 (2001).
168. S. Lewenza, D. Vidal-Ingigliardi, A. P. Pugsley, Direct visualization of red fluorescent lipoproteins indicates conservation of the membrane sorting rules in the family Enterobacteriaceae. J. Bacteriol. 188, 3516-24(2006).
169. W. T. Windsor et al., Disulfide bond assignments and secondary structure analysis of human and murine interleukin 10. Biochemistry. 32, 8807-15 (1993).
170. L. Thompson-Snipes et al., Interleukin 10: a novel stimulatory factor for mast cells and their progenitors. J. Exp. Med. 173, 507-10 (1991).
171. F. Sanchez-Munoz, A. Dominguez-Lopez, J.-K. Yamamoto-Furusho, Role of cytokines in inflammatory bowel disease. World J. Gastroenterol. 14, 4280-8 (2008).
172. Y. Shen et al., Outer membrane vesicles of a human commensal mediate immune regulation and disease protection. Cell Host Microbe. 12, 509-520 (2012).
173. B. M. Fournier, C. A. Parkos, The role of neutrophils during intestinal inflammation. Mucosal Immunol. 5, 354-366 (2012).
174. J. O. Lundberg, P. M. Hellström, J. M. Lundberg, K. Alving, Greatly increased luminal nitric oxide in ulcerative colitis. Lancet (London, England). 344, 1673-4 (1994).
175. R. K. Cross, K. T. Wilson, Nitric oxide in inflammatory bowel disease. Inflamm. Bowel Dis. 9, 179-89 (2003).
176. E. V Loftus, Clinical epidemiology of inflammatory bowel disease: incidence, prevalence, and environmental influences. Gastroenterology. 126, 1504-1517 (2004).
177. N. A. Molodecky et al., Increasing Incidence and Prevalence of the Inflammatory Bowel Diseases With Time, Based on Systematic Review. Gastroenterology. 142, 46-54.e42 (2012).
178. G. E. Crooks, G. Hon, J.-M. Chandonia, S. E. Brenner, WebLogo: a sequence logo generator. Genome Res. 14, 1188-90 (2004).
179. A.-C. Hochart-Behra, H. Drobecq, M. Tourret, L. Dubreuil, J. Behra-Miellet, Anti-stress proteins produced by Bacteroides thetaiotaomicron after nutrient starvation. Anaerobe. 28, 18-23 (2014).

### EQUIVALENTS

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. An engineered Bacteroides comprising a nucleic acid encoding a fusion protein comprising a Bacteroides membrane-associated protein linked to a heterologous protein.

2. The engineered Bacteroides of claim 1, wherein the heterologous protein is a therapeutic protein, a diagnostic protein or a prophylactic protein.

3. The engineered Bacteroides of claim 1, wherein the membrane-associated protein is truncated.

4. The engineered Bacteroides of claim 1, wherein the membrane-associated protein is a Bacteroides lipoprotein.

5. The engineered Bacteroides of claim 4, wherein the Bacteroides lipoprotein comprises a N-terminal signal peptide.

6. The engineered Bacteroides of claim 5, wherein at least 50%, at least 60% or at least 70% of the amino acid composition of the N-terminal signal peptide is aspartic acid (D).

7. The engineered Bacteroides of claim 5, wherein the N-terminal signal peptide comprises a cleavage site for a signal peptidase.

8. The engineered Bacteroides of claim 7, wherein the signal peptidase is signal peptidase I (SPI) or signal peptidase II (SPII).

9. The engineered Bacteroides of claim 7, wherein the cleavage site of the signal peptidase is within the first 15-50 amino acids of the Bacteroides lipoprotein.

10. The engineered Bacteroides of claim 1, wherein the Bacteroides membrane-associated protein is selected from the group consisting of: BT1491 proteins, BT3238 proteins, BACOVA_04502 proteins, and truncated variant proteins thereof.

11. The engineered Bacteroides of claim 10, wherein the Bacteroides membrane-associated protein is a N-terminal peptide of a BT1491 protein, a BT3238 protein, or a BACOVA_04502 protein.

12. The engineered Bacteroides of claim 1, wherein the therapeutic protein is an antibody, a cytokine or a growth factor.

13. (canceled)

14. The engineered Bacteroides of claim 12, wherein the growth factor is TGF-beta or keratinocyte growth factor, or wherein the cytokine is IL-2, IL-22, or IL-10.

15-16. (canceled)

17. The engineered Bacteroides of claim 1, wherein the membrane-associated protein is linked to the N-terminus of the therapeutic protein.

18. The engineered Bacteroides of claim 1, wherein the nucleic acid comprises a promoter operably linked to a nucleotide sequence encoding the fusion protein.

19. The engineered Bacteroides of claim 18, wherein the promoter is an inducible promoter.

20. The engineered Bacteroides of claim 1, wherein the Bacteroides membrane-associated protein facilitates the secretion of the fusion protein into the periplasm of the engineered Bacteroides, or facilitates the display of the fusion protein on the outer membrane of the engineered Bacteroides.

21. (canceled)

22. The engineered Bacteroides of claim 1, wherein the fusion protein is incorporated into a Bacteroides outer membrane vesicle (OMV).

23. The engineered Bacteroides of claim 1, wherein the Bacteroides is selected from the group consisting of: B. thetaiotaomicron, B. ovatus, B. fragilis, B. vulgatus, B. distasonis and B. uniformis.

24. An engineered Bacteroides comprising a fusion protein comprising a Bacteroides membrane-associated protein linked to a therapeutic protein.

25-43. (canceled)

44. An engineered Bacteroides outer membrane vesicle (OMV) comprising a Bacteroides fusion protein comprising a membrane-associated protein linked to a therapeutic protein.

45-68. (canceled)

69. An engineered nucleic acid encoding a fusion protein comprising Bacteroides membrane-associated protein or peptide linked to a therapeutic protein.

70. A method of administering to a subject the engineered Bacteroides of claim 1.

71-73. (canceled)


SEQUENCE LISTING (52 sequences)

SEQ ID NO: 1 - protein, 70 aa, Bacteroides thetaiotaomicron
MKAKMKKLSLFLLVSVFAFCGCSDDDEKMEVVISFENQLTEAESEFKTDLGEKGEVYFKYEISDPQKMIQ

SEQ ID NO: 2 - protein, 70 aa, Bacteroides thetaiotaomicron
MKKYIIMASVAALAIGLSSCSNFLDELPDNRTELNENNVGKILLSAYPTTAICEMGEMSSDNTDAYPNRF

SEQ ID NO: 3 - protein, 70 aa, Bacteroides ovatus
MKINKFLISGMLLMLGTSCSNDDTYTLCDECNGQKIIDITQFGLPTDGATDCADLINAIIADLPPEGGTI

SEQ ID NO: 4 - protein, 18 aa, Artificial Sequence
  Synthetic Polypeptide
MKAKMKKLSLFLLVSVFA

SEQ ID NO: 5 - protein, 19 aa, Artificial Sequence
  Synthetic Polypeptide
MKAKMKKLSLFLLVSVFAC

SEQ ID NO: 6 - protein, 24 aa, Artificial Sequence
  Synthetic Polypeptide
MKAKMKKLSLFLLVSVFACGCSDD

SEQ ID NO: 7 - protein, 42 aa, Artificial Sequence
  Synthetic Polypeptide
MKAKMKKLSLFLLVSVFACGCSDDDEKMEVVISFESEFKTDL

SEQ ID NO: 8 - protein, 43 aa, Artificial Sequence
  Synthetic Polypeptide
MKAKMKKLSLFLLVSVFAFCGCSDDDEKMEVVISFPQKMIQLS

SEQ ID NO: 9 - protein, 43 aa, Artificial Sequence
  Synthetic Polypeptide
MKAKMKKLSLFLLVSVFAFCGCSDDDEKMEVVISFKTPGYSNL

SEQ ID NO: 10 - protein, 20 aa, Artificial Sequence
  Synthetic Polypeptide
MKKYIIMASVAALAIGLSSC

SEQ ID NO: 11 - protein, 23 aa, Artificial Sequence
  Synthetic Polypeptide
MKKYIIMASVAALAIGLSSCSNF

SEQ ID NO: 12 - protein, 35 aa, Artificial Sequence
  Synthetic Polypeptide
MKKYIIMASVAALAIGLSSCSNFLDELPDNRTELN

SEQ ID NO: 13 - protein, 19 aa, Artificial Sequence
  Synthetic Polypeptide
MKINKFLISGMLLMLGTSC

SEQ ID NO: 14 - protein, 22 aa, Artificial Sequence
  Synthetic Polypeptide
MKINKFLISGMLLMLGTSCSND

SEQ ID NO: 15 - protein, 28 aa, Artificial Sequence
  Synthetic Polpeptide
MKINKFLISGMLLMLGTSCSNDDTYTLC

SEQ ID NO: 16 - protein, 42 aa, Artificial Sequence
  Synthetic Polypeptide
MKINKFLISGMLLMLGTSCSNDDTYTLCDECNGQPTDGATDC

SEQ ID NO: 17 - DNA, 60 bp, Artificial Sequence
  Synthetic Polynucleotide
ccaccgccaccgctaccgccaccgccagcgaatactgataccagtaagaataaagataac

SEQ ID NO: 18 - DNA, 60 bp, Artificial Sequence
  Synthetic Polynucleotide
tgccaccgccaccgctaccgccaccgccatcatcactacatccacaaaaagcgaatactg

SEQ ID NO: 19 - DNA, 60 bp, Artificial Sequence
  Synthetic Polynucleotide
tgccaccgccaccgctaccgccaccgcctaaatctgttttaaattcactctctgcttcag

SEQ ID NO: 20 - DNA, 60 bp, Artificial Sequence
  Synthetic Polynucleotide
atactgccaccgccaccgctaccgccaccgccagatagctgtatcatcttttgcggatcg

SEQ ID NO: 21 - DNA, 60 bp, Artificial Sequence
  Synthetic Polynucleotide
catactgccaccgccaccgctaccgccaccgccgaggttgctatagccgggagtctttac

SEQ ID NO: 22 - DNA, 20 bp, Artificial Sequence
  Synthetic Polynucleotide
ggcggtggcggtagcggtgg

SEQ ID NO: 23 - DNA, 60 bp, Artificial Sequence
  Synthetic Polynucleotide
cggattaaaaccaaaccctagaactaaaggttctggtcatcatcaccatcaccactaatg

SEQ ID NO: 24 - DNA, 60 bp, Artificial Sequence
  Synthetic Polynucleotide
tgttatttcagccggattaaaaccaaaccctagaactaaaggcggtggcggtagcggtgg

SEQ ID NO: 25 - DNA, 60 bp, Artificial Sequence
  Synthetic Polynucleotide
tggccattattatgtatttcttcatgtttatattatttatatttgtttgacgagaatatc

SEQ ID NO: 26 - DNA, 60 bp, Artificial Sequence
  Synthetic Polynucleotide
ctcgtcaaacaaatataaataatataaacaagaaatacataataatggccagtgtggccg

SEQ ID NO: 27 - DNA, 60 bp, Artificial Sequence
  Synthetic Polynucleotide
agtggtgatggtgatgatgaccagaacctttagttctagggtttggttttaatccggctg

SEQ ID NO: 28 - DNA, 60 bp, Artificial Sequence
  Synthetic Polynucleotide
tgccaccgccaccgctaccgccaccgcctttagttctagggtttggttttaatccggctg

SEQ ID NO: 29 - DNA, 60 bp, Artificial Sequence
  Synthetic Polynucleotide
caccgccaccgctaccgccaccgccacaaaaagcgaatactgataccagtaagaataaag

SEQ ID NO: 30 - DNA, 60 bp, Artificial Sequence
  Synthetic Polynucleotide
aaccatactgccaccgccaccgctaccgccaccgccacacgatgagagccctattgcgag

SEQ ID NO: 31 - DNA, 60 bp, Artificial Sequence
  Synthetic Polynucleotide
actgccaccgccaccgctaccgccaccgccgaaattagaacacgatgagagccctattgc

SEQ ID NO: 32 - DNA, 60 bp, Artificial Sequence
  Synthetic Polynucleotide
catactgccaccgccaccgctaccgccaccgccattcagctcggtacgattgtcgggaag

SEQ ID NO: 33 - DNA, 60 bp, Artificial Sequence
  Synthetic Polynucleotide
accatactgccaccgccaccgctaccgccaccgccgcaagatgtgcccaacatcaggagc

SEQ ID NO: 34 - DNA, 60 bp, Artificial Sequence
  Synthetic Polynucleotide
atactgccaccgccaccgctaccgccaccgccgtcattgctgcaagatgtgcccaacatc

SEQ ID NO: 35 - DNA, 60 bp, Artificial Sequence
  Synthetic Polynucleotide
tactgccaccgccaccgctaccgccaccgccacagagagtataggtatcgtcattgctgc

SEQ ID NO: 36 - DNA, 60 bp, Artificial Sequence
  Synthetic Polynucleotide
catactgccaccgccaccgctaccgccaccgccacaatcagtggccccatcggttggaag

SEQ ID NO: 37 - DNA, 60 bp, Artificial Sequence
  Synthetic Polynucleotide
aaggcggtggcggtagcggtggcggtggcagtatgtcacggggtcagtacagccgcgaag

SEQ ID NO: 38 - DNA, 60 bp, Artificial Sequence
  Synthetic Polynucleotide
tcttgacggtatcaccctcatcgaaaaaggcggtggcggtagcggtggcggtggcagtat

SEQ ID NO: 39 - DNA, 60 bp, Artificial Sequence
  Synthetic Polynucleotide
cggattaaaaccaaaccctagaactaaaggcggtggcggtagcggtggcggtggcagtat

SEQ ID NO: 40 - DNA, 60 bp, Artificial Sequence
  Synthetic Polynucleotide
tatattacattatctaagcgctaagaaaggcggtggcggtagcggtggcggtggcagtat

SEQ ID NO: 41 - DNA, 60 bp, Artificial Sequence
  Synthetic Polynucleotide
ctacatgatgattaaaatgaaaagctaaggttctggtcatcatcaccatcaccactaatg

SEQ ID NO: 42 - DNA, 60 bp, Artificial Sequence
  Synthetic Polynucleotide
tggtgatggtgatgatgaccagaaccgcttttcattttaatcatcatgtaggcttcaatg

SEQ ID NO: 43 - DNA, 28 bp, Artificial Sequence
  Synthetic Polynucleotide
gtgcagttcattagtggtgatggtgatg

SEQ ID NO: 44 - DNA, 60 bp, Artificial Sequence
  Synthetic Polynucleotide
attcgctttttgtggatgtagtgatgatggcggtggcggtagcggtggcggtggcagtat

SEQ ID NO: 45 - DNA, 60 bp, Artificial Sequence
  Synthetic Polynucleotide
gcttcccgacaatcgtaccgagctgaatggcggtggcggtagcggtggcggtggcagtat

SEQ ID NO: 46 - DNA, 60 bp, Artificial Sequence
  Synthetic Polynucleotide
cagcaatgacgatacctatactctctgtggcggtggcggtagcggtggcggtggcagtat

SEQ ID NO: 47 - DNA, 60 bp, Artificial Sequence
  Synthetic Polynucleotide
cttcttatttctgtcttgacggtatcaccctcatcgaaaaaggcggtggcggtagcggtg

SEQ ID NO: 48 - DNA, 60 bp, Artificial Sequence
  Synthetic Polynucleotide
atgttatttcagccggattaaaaccaaaccctagaactaaaggcggtggcggtagcggtg

SEQ ID NO: 49 - protein, 6 aa, Artificial Sequence
  Synthetic Polypeptide
HHHHHH

SEQ ID NO: 50 - DNA, 60 bp, Artificial Sequence
  Synthetic Polynucleotide
tcatgacgaagaatatattacattatctaagcgctaagaaaggcggtggcggtagcggtg

SEQ ID NO: 51 - DNA, 35 bp, Artificial Sequence
  Synthetic Polynucleotide
gtttatattatttatatttgtttgacgagaatatc

SEQ ID NO: 52 - DNA, 60 bp, Artificial Sequence
  Synthetic Polynucleotide
aaagatattctcgtcaaacaaatataaataatataaacatgtcacggggtcagtacagcc